(19) United States
(12) Patent Application Publication
(10) Pub. No.: US 2009/0053689 A1
(43) Pub. Date: Feb. 26, 2009

(54) DEVICE FOR PROCESSING A BIOLOGICAL AND/OR CHEMICAL SAMPLE AND METHOD OF USING THE SAME

(75) Inventors: Dominador Fortaleza Oviso, JR., Singapore (SG); Dor Ngi Ting, Singapore (SG); Farid Ghadessy, Singapore (SG); Hui Wang, Singapore (SG); Wen Jian Lau, Singapore (SG)

Correspondence Address: Biotechnology Law Group;c/o Portfolioip, P.O. Box 52050, Minneapolis, MN, 55402, US

(73) Assignee: ATTOGENIX BIOSYSTEMS PTE. LTD, Singapore (SG)

(21) Appl. No.: 11/996,237

(22) PCT Filed: Jul. 19, 2005
(86) PCT No.: PCT/SG05/00241

§ 371 Date: Sep. 26, 2008

(51) Int. Cl. C12Q 1/70; B01L 3/00; G01N 1/28; C12Q 1/02; G01N 33/04; G01N 33/24; C12Q 1/68; B01L 11/00; G01N 33/48; G01N 33/68; G01N 33/92; G01N 33/00; G01N 33/44

(52) U.S. Cl. 435/5; 422/102; 436/174; 435/29; 436/23; 436/25; 436/63; 436/64; 436/86; 436/71; 436/94; 436/89; 436/85; 435/6; 422/101

(57) ABSTRACT

The present invention relates to a device and an apparatus device for processing a biological and/or chemical sample. The device comprises at least one sample processing chamber having an inlet at a first end and a penetrable sealing layer at a second end that forms at least a part of an inner wall of the sample processing chamber. The sealing layer is adapted to seal off the sample processing chamber from the environment until said sealing layer is penetrated to form an outlet. The device and apparatus also includes an absorption layer, wherein upon penetration of said sealing layer, said absorption layer is in fluid contact with said sealing layer and is capable of absorbing fluid released from said sample processing chamber, via the outlet. The present invention also relates to a fluid separation device. The fluid separation device comprises a penetrable sealing layer adapted to form a wall of a sample processing chamber of the device of the invention. The fluid separation device also comprises an absorption layer that is in fluid contact with the sealing layer and capable of absorbing fluid released from a sample processing chamber of the device.

### FIELD OF THE INVENTION

The present invention relates to a device for processing a biological and/or chemical sample, more specifically, to a device for carrying out chemical and biological processes on a sample and a method of using said device.

### BACKGROUND OF THE INVENTION

The processes of separation, isolation and further purification of sample components from various starting materials is of great importance for a many laboratories working in the field of biotechnology, laboratory medicine, biochemistry, veterinary medicine, food analysis, petrochemicals, chemical synthesis and other related fields.

In the organic synthesis industry, attempts to minimize systems of chemical reactors have so far been hampered by the lack of available separation means, which can be combined with a reactor. Correspondingly in the life science industry, there is also often need for fast isolation means for a sample material due to the risk of decomposition or degradation and reliable means to prevent sample contamination. In addition, both the above-mentioned industries of science utilize common analysis methods that often require the removal of certain components, such as salts or detergents.

In order to support the above-mentioned rapidly growing industries, new techniques and methodologies are typically developed in tandem with new technical advances. These developments (both technical and methodological) generally aid in the analysis and investigation of the constituents of various cellular forms. Analysis at this level is often carried out through the investigation of a captured nucleic acid, such as for instance deoxyribonucleic acid (DNA) and ribonucleic acid (RNA), or a protein for example originating from an organism or a cell type. In addition, to better understand the organism (or cell-type), or to isolate a compound of interest, a respective compound or organism is typically extracted using physical, chemical and/or biological interactions with different probes, chemicals, probes, and/or enzymes.

Extracting nucleic acids from various materials is known to be a tedious process. A recent evaluation report (Mattocks C, “Automated extraction methodologies”, www.ngrl.org uk/Wessex/downloads/Evaluation_of_Automated_Extraction_Methodologies_{—}093444.pdf) gives an overview on current methods used to extract nucleic acids. The following patent applications describe devices that serve to aid in the extraction of DNA and RNA.

U.S. Pat. No. 6,649,419 describes a method and an apparatus for extracting, identifying and manipulating proteins or peptides using magnetic beads. The magnetic beads include a coating having an affinity to absorb/adsorb proteins and peptides and hence, the magnetic beads are used to separate and purify, immobilize and assay antibodies. Once the magnetic beads are bound to their respective protein or peptide, a magnetic rod is inserted to attract said beads to the rod. The binding reactions between the beads and the desired protein or peptide, and any subsequent processing reactions take place in several individual chambers and require the magnetic beads to be removed from one chamber and be physically transported, with environmental exposure, to the next chamber for further processing. Accordingly, there lies a risk of the sample or the target matter being contaminated.

US patent application 2004/0126783 describes an apparatus having a porous support, wherein said porous support includes an agent that reacts with a nucleic acid inhibitor component upon contact. The apparatus also includes a housing with a chamber defined therein. The nucleic acid component is directed through a portion of the porous support and later on, is separated from said support. The separation means of the nucleic acid from the support is carried out by a magnetic substrate means. The magnetic substrate binds to the nucleic acid and is later extracted from the sample by means of a magnetic field provide for by a magnet.

In addition to the above devices, European Patent EP 0389063 describes a method of isolating and purifying a nucleic acid from lysates of various samples. The method disclosed in EP 0389063 involves the mixing of the said lysates with a chaotropic substance and a particulate nucleic acid binding solid phase comprising silica or a derivative thereof. It is known that, in the presence of a chaotropic substance, nucleic acids are released from cells and cell lysates, and bind to silica-based nucleic acid binding solid phases. This method commonly utilizes layers of matrices of silica fibers as a binding media, and subsequent ultra-centrifugation steps are required to separate the supernatants after each washing stage. In some embodiments of the method, the centrifugation step can be eliminated, by use of commercially available silica-coated magnetic beads for binding with nucleic acid. Subsequently after binding with the nucleic acids, the sample and magnetic beads mixture are subjected to a magnetic field in which the supernatants are separated. Further examples of apparatuses for processing a biological sample have been disclosed in U.S. Pat. No. 6,562,239, U.S. Pat. No. 6,413,420, US patent application 2004/0173537 and US patent application 2005/0009071.

In each of the above-mentioned methods and apparatus used in the processing of test samples, the difficulty of preventing contamination arises. Recent advances promote the use of automated systems to perform the processing of the test sample. An example of such a system utilizes functionally coated magnetic beads for specific separation of nucleic acids, for example. However, the present systems that utilize magnetic beads in biological and/or chemical sample processing require either multiple transferences of the sample or reagent, or are unable to carry out several processing steps simultaneously or sequentially.

Accordingly, despite the above-mentioned devices and method, there still exists a need for a sample-processing device that is simple to use, capable of providing contamination-free processing of single and multiple samples, is scalable in size and yet cost-effective to produce. Such a device, and apparatus, as defined in the independent claims appended herewith, overcomes the above-mentioned difficulties.

### SUMMARY OF THE INVENTION

The present invention provides a device for processing a biological and/or chemical sample, more specifically, to a device for carrying out one or more chemical and/or biological processes on a sample and a method of using said device. Thus in one aspect the present invention provides a device for processing a biological and/or chemical sample. The device for processing a biological and/or chemical sample includes at least one sample processing chamber having an inlet at a first end and a penetrable sealing layer at a second end. The sealing layer forms at least a part of an inner wall of the sample processing chamber. The sealing layer is adapted to seal off the sample processing chamber from the environment until said sealing layer is penetrated to form an outlet. The device further includes an absorption layer, wherein upon penetration of said sealing layer, said absorption layer is in fluid contact with said sealing layer. The absorption layer is capable of absorbing fluid released from said sample processing chamber, via the outlet.

In another aspect the invention provides an apparatus for processing a biological and/or chemical sample. The apparatus includes at least one device as described above and a probe. The probe is adapted to selectively attract target matter from the sample processing chamber.

In a further aspect the invention provides a method of processing a biological and/or chemical sample using the device described above. The method includes:
(a) Providing a device for processing a biological and/or chemical sample as described above;
(b) Providing biological and/or chemical sample;
(c) Placing the sample a into the sample processing chamber of the device, thereby forming a reaction mixture.

In yet another aspect the invention provides a further method of processing a biological and/or chemical sample. The method includes:
(a) Providing an apparatus for processing a biological and/or chemical sample as described above,
(b) Providing a biological and/or chemical sample;
(c) Placing the sample a into the sample processing chamber of a device of the apparatus, thereby forming a reaction mixture;
(d) Exposing the reaction mixture to a process, thereby forming a product mixture;
(e) Selectively attracting target matter suspected to be comprised in the product mixture to the surface of the probe; and
(f) Using the target matter, with or without release from the probe, for further use such as for storage, detection, analysis and disposal.

In yet another aspect the invention provides a fluid separation device. The fluid separation device includes a penetrable sealing layer and an absorption layer. The penetrable sealing layer is adapted to form a wall of a sample processing chamber of the device as described above. The absorption layer is in fluid contact with said sealing layer upon penetration of the sealing layer. Upon penetration of the sealing layer the absorption layer is capable of absorbing fluid released from the sample processing chamber, via the outlet.

### DETAILED DESCRIPTION OF THE INVENTION

The device for processing a biological and/or chemical sample includes at least one sample processing chamber having an inlet at a first end and a penetrable sealing layer at a second end. The sealing layer is adapted to seal off the sample processing chamber from the environment until the sealing layer is penetrated to form an outlet.

The term “environment” refers to the exterior (vs. the interior) of the sample processing chamber and therefore means any matter that is not located inside or contacting the interior of the sample processing chamber. It thus refers to any space or matter that is not in direct contact with fluid filled into the sample processing chamber while the sealing layer is intact. They are consequently not facing the environment. Hence, the environment includes further elements of the device. One such element are one or more absorption layers. At least one respective absorption layer is therefore upon penetration of the sealing layer in fluid contact with the sealing layer. In some embodiments at least one such absorption layer is directly contacting the sealing layer, for instance by means of direct physical contact. In some embodiments the sample processing chamber, the sealing layer and the absorption layer form one physical unit. In some of these embodiments both the sealing layer and the absorption layer form a wall or a part of a wall of the sample processing chamber. The absorption layer may for example be integrated into the sealing layer or arranged between two sealing layers or a sealing layer and a supportive layer. An absorption layer is capable of accommodating, soaking up, incorporating, sucking in, transmitting or otherwise absorbing or accepting fluid released from the sample reaction chamber, via the outlet that is created upon penetration of the sealing layer.

As a chamber is defined to be such that it is cut-off from the external environment as defined above, it follows that the sealing layer is affixed to the chamber in a manner to maintain a desired fluid, for instance a liquid, in the sample processing chamber, i.e. to remain impermeable for the fluid contained therein. In some embodiments the sealing layer hermetically seals the sample processing chamber from the environment. In other embodiments the sealing layer is permeable for fluid that is not of interest for a desired process to be carried out in the sample processing chamber, such as for instance gas.

In one embodiment, the sealing may occur by way of adding adhesives or sealing gels, for example to the periphery of the sealing layer to obtain an airtight fit between the sealing layer and the chamber. In other embodiments, the sealing layer may be attached to the chamber either mechanically (by pins or stitching) or by a snap-fit or tight-fit mechanism.

Accordingly, when the sealing layer is affixed to the chamber, the sealing layer forms an inner wall of or a portion of an inner wall of the chamber. As indicated above, the interior of the sample processing chamber means any space or matter that is in direct contact with fluid filled into the sample processing chamber while the sealing layer is intact. It also refers to any space or matter that may be comprised in space or matter contacting such fluid. Accordingly the term “inner wall”, when used in connection with the sample processing chamber, refers to surface areas that face the interior of the sample processing chamber in that they are able to contact fluid filled therein while the sealing layer is intact. The chamber is then capable of receiving a fluid and/or a sample, e.g. a chemical and/or biological sample, as well as optionally further matter. Hence, in some embodiments the sample processing chamber is filled with a medium, for instance a medium for carrying out a chemical and/or biological process. In other embodiments it may be desired to fill the sample processing chamber with such medium in order to carry out a chemical or biological process. The sealing layer thus acts to prevent any fluidic communication between the interior of the chamber and the surrounding environment.

As indicated above, in some embodiments the sealing layer and the absorption layer may form one common unit. They may for instance form a sealing portion, which may as a whole be penetrable. A respective sealing portion may form at least a part of a wall of the sample processing chamber. A part of or the entire inner wall of the sample processing chamber may thus for example be formed by the sealing layer comprised in a respective sealing portion. The external surface of the sealing layer may thus be adapted to seal off the sample processing chamber from the environment as defined above, until it is penetrated to form an outlet. The external surface of the sealing layer is defined in that it does not contact any fluid filled into the sample processing chamber while the sealing layer is intact, i.e. not forming an outlet. Hence, the external surface of the sealing layer is in fluid contact with the absorption layer. In some embodiments it is in direct physical contact with the absorption layer. In one of these embodiments the sealing layer forms a housing for the absorption layer. Any such embodiment of a combined sealing layer and an absorption layer may form a fluid separation device that is adapted to be used with a sample processing chamber of a device of the present invention. Such a fluid separation device is thus adapted to absorb fluid released from a sample processing chamber. It is furthermore adapted to complete a device as described above, when combined with the sample processing chamber.

Where fluid, e.g. liquid, is desired to exit the sample reaction chamber, the sealing layer will be penetrated. The penetrability of the sealing layer depends upon the properties of the material used to fabricate said sealing layer. For instance, a material with a high tensile strength or elasticity will typically have a low degree of penetrability. As the sealing layer should be easily penetrable by a user, the tensile strength of the material used should generally not be of not too high strength. The sealing layer may have, in addition to generally being of lower tensile strength, several perforations or break lines to aid in the penetration of the sealing layer. The perforations may also serve as pre-existing cracks within the structure of the sealing layer to further aid in the penetration of said sealing layer by the user.

If present, the perforations on the sealing layer are typically such that they do not run through from the internal surface to the external surface of the sealing layer in order to preserve the integrity of the sealed chamber and to prevent fluid communication between the internal surface and external surface. Instead, the perforations may, for example, only extend from the internal surface to half (or alternatively to three-quarters) of the thickness of the sealing layer. Perforating a given portion (or region) of the sealing layer causes said portion to be structurally weaker as, at a macroscopic level, some of the bonding mechanisms may be weakened. This allows the integrity of the sealed chamber to be maintained whilst easing the possible penetration of the sealing layer. Such a configuration may be particularly useful in embodiments where the sealing layer is penetrated by a probe.

Alternatively, the penetrable sealing layer may include a one-way valve. A respective valve may for instance be operable by mechanical or magnetic means. Other means to operate a respective valve include, but are not limited to, pressure, a change in temperature, and/or a change in chemical property such as pH in cases where a smart material is used (see below). As an illustrative example, a valve may include one or more elements of piezoelectric material (see below). A valve may furthermore be adapted to permit only a probe to pass through the sealing layer. In an embodiment including said one-way valve, dimensions of the valve may be such that it allows for a semi-tight fitting between the interface of the probe and that of the valve. The semi-tight fitting is necessary to allow the probe to continue to slide through said valve. Accordingly, it is to be understood that in this embodiment, the one-way valve functions, essentially, as an outlet for the chamber.

In further embodiments the sealing layer includes one or more elements that are able to disintegrate or that cause other parts or elements of the sealing layer to disintegrate. Such elements form an outlet or generate an outlet. This may be achieved by means including, but not limited to, pressure, mechanical means, moisture, an electrical field, a magnetic field, a change in temperature, and a change in pH of the fluid comprised in the sample processing chamber, or any combination thereof. In some embodiments respective properties of a sealing layer are achieved by including a smart material. The term “smart”, or responsive, material is used in the art for materials that react to their external environment by bringing on a desired response. These materials thus possess a property that can be significantly altered in a controlled manner. External factors that may be suitable to alter respective properties include stress, temperature, moisture, pH, electric or magnetic fields. Any respective material can be included into the sealing layer of the present invention. Examples of suitable smart materials include, but are not limited to, piezoelectric materials (see below), thermoresponsive materials, shape memory alloys, shape memory polymers, and pH sensitive materials.

Shape memory alloys and shape memory polymers can hold different shapes at various temperatures. An illustrative example of a respective polymer is Pluronic F127. Solutions of 14-30% Pluronic F127 are fluids at low temperatures and gels at room temperature. As a further example, an aqueous solution of a poly(ethylene glycol-b-[DL-lactic acid-co-glycolic acid]-b-ethylene glycol) (PEG-PLGA-PEG) triblock copolymer forms a free-flowing sol at room temperature and becomes a gel at body temperature. A further example are phenylboronic acid-poly(vinyl alcohol) hydrogels, which swell when glucose binds to them.

An illustrative example of a pH sensitive material is poly(2-hydroxyethyl-methacrylate) (PHEMA), which swells in basic solution and shrinks in acidic solution. An example of a material that is sensitive to an electric field is a semi-interpenetrating polymer network (semi-IPN) hydrogel, composed of chitosan and poly(hydroxyethyl methacrylate) (PHEMA, see above). When in contact with aqueous NaCl solutions, this material swells. If the material is exposed to an electrical field in this state, it shows a bending behaviour. Two illustrative examples of a temperature sensitive material are poly(N-isopropylacrylamide) (pNiPAAm) and poly(N,N-diethylacrylamide) (PDEAAm). These materials undergo a reversible swelling within a defined temperature range. The swelling is accompanied with a change in volume and hydrophobicity. The change of pNiPAAm is further sensitive to the pH of a solution, which is in contact with it. Where desired, some smart materials such as pH-sensitive materials or chromogenic materials may also be used for indicator purposes in the device of the invention. Chromogenic materials change colour in response to electrical, optical or thermal changes.

As an illustrative example for a respective sealing layer, the sealing layer may contain a wax. Such a wax may melt when subject to its respective melting temperatures. As a further illustrative example, is for instance common in the art to characterize polymers by the dependence of their viscoelastic properties on the temperature. Typically mechanical properties of a polymer change at the crystalline melting temperature. As an illustrative example it may be desired to perform a process in the device at a certain temperature. Given this temperature, polymers can be identified, which posses a crystalline melting temperature close to the chosen temperature. A part of the sealing layer may then consist of this polymer. The polymer will stand the reaction conditions, however a further change in conditions may easily cause the polymer to change its aggregation state. After the process has reached a desired degree of completion, a change in pressure may for instance be applied to cause an opening of the sealing layer at the respective site. An example of a polymer of a very low crystalline melting temperature is polydimethylsiloxane with a Tₘ of −40° C. An example of a polymer with a crystalline melting temperature close to room temperature is natural rubber (polyisoprene) with a Tₘ of 28° C. As yet another example, a thin layer of an intrinsically brittle polymer, such as, but not limited to, polystyrene and poly(vinyl chloride), is easily susceptible to pressure or mechanical force. Where desired, a so called “rigid-rod” polymer may be employed. Rigid-rod polymers are polymers, which do not undergo a change of polymer conformation, although the primary backbone of the polymer is deformed by applied stress. Examples of a device possessing a sealing layer that is able to disintegrate in its entirety are depicted in FIG. 16 in use.

If present, the shape of perforations may vary according to the shape of the probe being utilized to create the outlet. Should it be desired to use a probe which is generally cylindrical in nature, an asterisk shape perforation may be used (cf. e.g., FIGS. 3a to 3c), as it would also provide circumferential support to the probe as an added advantage, for example.

Alternatively, a hinged design may be employed in that instead of having a complete perforation of a square, for example (as depicted e.g. in FIG. 3D), only three of the four sides are perforated with the fourth side left intact (as e.g. depicted in FIG. 3F). In such an embodiment, the resulting penetration would cause the three perforated sides to detach from the sealing layer and bend about the remaining side (not perforated) that is still attached to the rest of the sealing layer.

Using the above-mentioned embodiment of a square with three perforated sides, a further alternative may be to have a notch with the acute portion of said notch extending upwards from the external surface of the sealing layer towards the internal surface (cf. FIGS. 3J, 3K and 3L). By having such a design, a hinge-like mechanism for the penetrated portion of the sealing layer provides for and allows the penetration portion to pivot about said hinge. The penetration portion is able to remain securely within the original sealing layer. The usage of perforated lines in this embodiment enables the user to pre-locate or define the location of the hinge. This in turn may aid in the perforated sides providing a means of controlling the size of the opening in the sealing layer during penetration. The selection of such embodiments may often provide the advantage that the risk of having a portion of the sealing layer fracturing or detaching and contaminating the next processing step is reduced.

In some embodiments the sealing layer contains a recess. The recess may be of a larger diameter than the spatial extent of the penetrating portion of the probe in the same dimension. In some of these embodiments, the recess is present on the side of the sealing layer that forms part of the internal wall of the chamber. The recess may be such that it contains only a very small volume of fluid. The recess may, for example, be selected to be able to accommodate a volume of less than 10 μl. The small recess may also be adapted to fit to the probe such that when the probe is inserted into the chamber and over the recess, the probe may form, in conjunction with the recess, a mini-reaction chamber within said chamber. (cf. FIG. 11A).

The sample reaction chamber may include any desired material. Typically, walls of the sample reaction chamber are solid and able to remain intact during the entire process to be performed therein. The sample reaction chamber may be of any desired volume. It may for example be able to accommodate a volume of 0.1 μl to 100 ml, or a volume of 1 μl to 10 ml, or 10 μl to 2 ml. Where fluid is filled into the chamber, only part of the volume of the sample reaction chamber may be filled with the respective fluid. The remainder of the chamber volume may for instance be occupied by other fluid, such as air or an inert gas.

The inlet of the sample processing chamber may consist of or may include any means. As an illustrative example, the inlet may be an opening. Such an opening may be of any size and shape. Examples include, but are not limited to, the shape of a circle (cf. e.g. FIGS. 2A, 2C, 2D, 2E or 2F), rectangular or square shape (cf. e.g. FIG. 2B or 2H) or the shape of a triangle (cf. e.g. FIG. 2G). In all embodiments described herein, the inlet of the chamber may be entirely sealable. The sealing of the inlet may be achieved by means of a lid. Other sealing means include, but are not limited to a rubber gasket or a self-sealing polymer membrane. In the case of the latter, the self-sealing membrane, such as for instance polyurethane or silicone, allows hypodermic syringes, for example, to inject additional test samples, chemical reagents and biological additives such as enzymes to the chamber during various stages of the processing. In some embodiments the inlet is sealed by a penetrable sealing layer as described above. The inlet of the sample processing chamber may be located anywhere within the device. As an illustrative example, it may be located coaxially opposing the sealing layer.

In some embodiments the inlet of the sample processing chamber is adapted to receive a probe. As an illustrative example, the inlet may be a hole in a wall of the sample processing chamber, which is adapted to allow for a probe to enter. In such embodiments an interface, for instance a void space, may remain between the probe and a brim or edge of a respective hole. Such an interface between the probe and the wall of the chamber, covering the remaining size of the inlet, may be sealed by means of a sealant such as a rubber ring, a viscous gel or an epoxy material. Essentially, any sealing means that permits the probe to slide into the chamber and penetrate the sealing layer may be used.

When used for the processing of matter (e.g. a sample), the device may be subjected to an energy source for particular processes to be initiated or catalyzed. Examples of energy that may be applied, include, but are not limited to, microwave or photolytic energy. Accordingly in a further embodiment, the chamber of the device may be adapted to have a surface with waveguides that transmit the microwave or photolytic energy from the source towards the sample in the sample processing chamber. In addition to the above, in some embodiments piezoelectric materials are attached to the side walls of the chambers. Piezoelectric materials reversibly acquire a charge when compressed, twisted or distorted. Conversely they therefore change dimensions in a reversible manner, when exposed to an electric field, an effect termed electrostriction. These properties can be utilised for the generation of motion, respective devices are termed electro-mechanical transducers or piezo actuators. Examples of suitable piezoelectric materials include, but are not limited to, berlinite (AlPO₄), gallium orthophosphate (GaPO₄), barium titanate, lead titanate, lead zirconate, lead zirconate titanate (Pb[Zr,Ti]O₂), lead magnesium niobate, and polyvinylidene fluoride (—CH₂—CF₂—)ₙ.

The device may be configured for the processing of only one or a plurality of matter, such as for instance test samples, at the same time. In this respect, the sample processing chamber may be configured to accommodate or contain sub-chambers separated from each other and to be loaded with any target matter, e.g. one or more test samples. In some embodiments the device is adapted to provide a particular interior geometry for a desired reaction, for instance for the exposure of a plurality of test samples or only one sample to microwave or photolytic energy. The configuration aims to achieve a uniform distribution of the microwave or the photolytic energy for uniform photo activation or uniform heating of the contents of the chamber, for example.

In some embodiments the sample processing chamber may be adapted to include one or more sub-chambers (such as for instance a basket), which may for example be loaded with one or more test samples. In embodiments where the sample processing chamber includes such sub chamber(s), these sub-chamber may have a sealing layer as described above. In such an embodiment, the sub-chamber may be placed within the sample processing chamber along with a sample and/or requisite reagents for the reaction to be carried out. Accordingly, a first reaction may be carried out in said sub-chamber and the resulting contents of said first reaction may then be transported (via the penetration of the sealing layer) to the sample processing chamber of the device for further processing. A sub-chamber may located anywhere within a sample processing chamber. As two illustrative examples, they may be stacked within the sample processing chamber as shown in FIG. 4J, or side by side as shown in FIG. 4K. In this regard, it may also be possible to have a plurality of sub-chambers within a single sample processing chamber to provide for multiple processes within said single sample processing chamber. In some cases such an arrangement may be advantageous as it may allow for multiple reactions to take place while the device remains relatively compact.

In some embodiments the sample processing chamber has a thermostatic device or a Peltier heater incorporated therein to influence (by way of acting as a catalyst, for example) the processing of the chemical or biological sample by varying the temperature. In this regard, the thermostatic device or Peltier heater may act a heating means, a cooling means or both. Accordingly, the heating or cooling means may be, but is not limited to, a peltier heater attached to the surface or surfaces of the chamber, or a heating or cooling coil wound around the circumference of the chamber, for example to provide for uniform temperature distribution. A respective thermostatic device may be located anywhere in the sample processing chamber. Alternatively, when the device of the invention is used in connection with a probe, such a probe may include the thermostatic device.

In further embodiments, the device is adapted to be securable to at least one other device, such that the outlet of one device is in fluid communication with the inlet of another device. The securing means between any two devices may for instance be achieved by having corresponding slots and rails by which one device may be secured to another. Alternatively, a socket and plug configuration may be utilized as well. In the socket and plug embodiment, the base (or any other) portion of the device may include a socket. The corresponding or mating surface is thus capable of functioning as a plug, for example. A further alternative in securing at least two devices together may be achieved by using snap-fit or tight-fit means. In yet other embodiments one device is screwed onto another. Further methods of securing at least two such devices together may be used provided the guiding principle of maintaining the outlet of one device in fluid communication with the inlet of a subsequent device is adhered to. It should be understood that a single device of the present invention may furthermore contain more than one sample processing chamber (cf. e.g. FIG. 6). It may also include any additional element, for instance an elution chamber (cf. below). A person skilled in the art will appreciate that a device of the present invention avoids the exposure of matter to the atmosphere or ambience, while matter is transferred from one device or apparatus to another, and that the device thus avoids the risk of contamination (cf. FIG. 1).

For each of the above-mentioned securing means, an arrangement of a plurality (which refers to two or more) of devices may be carried out in series in any (one or more) dimensions. As an illustrative example, devices may be arranged vertically or horizontally. In a vertical arrangement, essentially, stacking takes place. The stacking may be collinear or in a step-like manner. Should a horizontal arrangement be used, again, the arrangement may be collinear or step-like but in a horizontal orientation. Illustrative embodiments of horizontal and vertical arrangements are depicted in FIG. 5. In a vertical arrangement, gravity may be relied on to play a major role in causing the discharge of fluid from the outlet, however, in the horizontal orientation, gravity typically plays a smaller role in the discharge. Depending upon the characteristics of the fluid in the chamber, various means may be used to move matter such as fluid through the outlet after the sealing layer is penetrated. Such possible means may be, but are not limited to, the utilization of a pressure difference, mechanical means, an electrical charge difference or a temperature difference between the outlet and the inlet of the subsequent chamber.

In stacking the devices, as mentioned above, alternatively an external modular mechanism may be used. Apart from supporting the stack, the external modular mechanism may also be adapted to function in conjunction with additional means used in a desired process. An illustrative example is medical diagnostic equipment. The external modular mechanism may for instance have an opening or slot adapted to accommodate the device of the present invention. Where for instance some medical diagnostic equipment is comprised in the external modular mechanism, such equipment may for example allow the user to select a suitable probe, which includes certain characteristics (such as for instance antibody coatings). It may furthermore allow a user to insert such suitable probe to perform a specific test. The external modular mechanism may also include a security mechanism that assists in securing the position of stacked devices to other medical devices.

In any of the embodiments described herein, the sealing layer may be adapted to be penetrable by mechanical means. In this regard, the above mentioned perforations may be used to adapt said sealing layer to be penetrable by mechanical means.

In other embodiments, the penetrable sealing layer and/or the absorption layer may be detachable. The sealing layer may be detachable from the outlet. It may furthermore be replaceable with at least one other penetrable sealing layer. The absorption layer may be detachable from the sealing layer. It may furthermore be replaceable with at least one other absorption layer. In such embodiments, the sealing layer may be adhered to the chamber or the absorption layer to the sealing layer by means of, but not limited to, a removable adhesive such as double-sided tape, a temperature dependent epoxy adhesive or an adhesive putty such as that from 3M (3M Scotch Removable Adhesive Putty), for example. Alternatively, the sealing layer may also be adhered to the chamber or the absorption layer to the sealing layer by mechanical means such as screw on adaptation, latches, tight-fitting or snap-fitting, for example. A respective replacement of a sealing layer or an absorption layer may be desired where a device that has already been used for performing a process therein is desired to be reused.

A detachable sealing layer or absorption layer may be advantageous in a number of uses of the device. This may for instance be due to the fact that the user may simply replace a sealing layer, which may for instance be penetrated or disintegrated with a new sealing layer. Likewise an absorbing layer, which may for instance be soaked with a fluid, may be exchanged for a new respective sealing layer. Where desired, this enables the user to reuse the chamber thereby reducing waste and costs.

As indicated above, in other embodiments the device may further include a second penetrable sealing layer. In some of these embodiments the first and the second sealing layer are arranged to sandwich the absorption layer between them. The second sealing layer may provide additional mechanical support to the absorption layer sandwiched there between. In some applications, the second sealing layer can also serve as a barrier to prevent contact of the absorbed fluid in the absorbing layer from in contact with the atmosphere particularly when the fluid maybe health hazard. The second sealing layer, as in the first sealing layer, may also be adapted to be penetrable by means of the above-mentioned perforations.

As indicated above, in some embodiments of the device, the penetrable sealing layer includes a recess. A head of a probe may enter into such a recess up to the given depth of said recess without penetrating the sealing layer (cf. FIGS. 11A-F and 11H). The depth of the recess can be of any size or depth. In one embodiment, the depth of the recess ranges between 0.5 mm to 10 mm, with a diameter of at least 0.5 mm. The usage of a respective recess depends both on the geometric properties of the sealing layer and on the probe involved in a selected process (see below and e.g. FIG. 11). As an illustrative example, where a probe includes a head and additional elements such as arms or blades etc., the head may jut further than such additional elements (cf. e.g. FIG. 11G). In such embodiments the volumes involved may be selected of very small quantity.

The sealing layer(s) of the device of the invention may comprise any material that is able to perform a sealing function under the conditions intended for its use. Where desired, the material may be selected to withstand certain reaction conditions that are intended to be used. Where for instance corrosive matter is used in a process performed in the sample processing chamber, the surface of the sealing layer that faces the interior of the sample processing chamber may be equipped with or consist of material that is inert to such matter. In other embodiments the sealing layer may be used to terminate a reaction. As an illustrative example a sealing layer of a certain material and with a certain strength may be known to disintegrate under selected reaction conditions after a defined period of time. In this case the strength of the sealing layer may be adapted to stand a desired time interval, where after it disintegrates thus releasing fluid from the sample processing chamber. This may cause the termination of a reaction occurring in the sample processing chamber.

Examples of materials that are typically suitable for the use of aqueous fluids include, but are not limited to, inorganic materials and natural or synthetic polymers. Examples of suitable polymers include, but are not limited to, polypropylene, polyisoprene, polystyrene, polyvinyl chloride, polyisobutylene, polyethylene terephthalate (PET), polyacrylates (e.g. polymethyl-methacrylate (PMMA)), ethylene-vinyl acetate (EVA) copolymers, phenol formaldehyde resins, epoxy resins, poly(N-propargylamides), poly(O-propargylesters), and polysiloxanes. Two illustrative examples of inorganic materials that may be used under most conditions are glass and a metal foil. A respective metal foil may for instance be an aluminium foil. A metal foil may optionally be coated with a polymer layer such as polyethylene or polypropylene, for example were it is desired to avoid oxidation.

The absorption layer may be designed to accommodate, soak up, incorporate, suck in, transmit or otherwise accept or absorb any volume of a selected fluid. It may thus for instance be designed to absorb any part or the complete volume of the sample processing chamber as well as of any other chamber other of the device. The absorption layer is in some embodiments furthermore designed to be penetrable.

In one embodiment, the absorption layer of the device may include at least two sub-absorption layers. In one embodiment having two absorption layers, the second absorption layer may be the same as the first in order to further increase the absorbance capacity of the absorption layer as a whole. In the embodiment having two similar absorption layers, should the volume of medium be too large for the first absorption layer to absorb completely, the subsequent absorption layer may also aid in absorbing any excess fluid not already absorbed by the first absorption layer, for example.

Alternatively, the absorption layer may include sub-absorption layers of varying absorptivity. The varying absorptivity may be of advantage when the medium contains fluids of varying volatility, viscosity or miscibility. If the medium contains two immiscible fluids, for example, the first absorption layer may be only capable of absorbing one of the two fluids leaving the second absorption layer to absorb the remaining fluid. Accordingly, two fluids of varying volatility may also makeup the medium. As above, it may be the case that a particular absorption material is used to absorb only one of the two fluids, for example.

As mentioned above, an alternative embodiment of the absorption layer utilized may have an affinity or a selectivity for absorbing particular fluids. Accordingly, in such an alternative embodiment the adsorption layer may be fabricated from a hydrophilic material or a hydrophobic material. Such material may for instance be used in form of a fluid-absorbing polymer powder or a polymer gel. In embodiments where the absorption layer is fabricated from a hydrophilic material, the hydrophilic material may for instance be based on a polysaccharide such as for instance cellulose. Such cellulose-based materials may be selected from, but are not limited to, the group consisting of a wood pulp fiber, a cotton linter, rayon fibers, flax, hemp, jute, ramie and cotton, for example. A cellulose fiber material well known in the art is commercially available as Whatman paper. Other fluid-absorbing polymers that may also be used in the fabrication of the absorption layer include, but are not limited to, cellulose, starch, polyurethane foam and poly(vinyl amine)s. Examples of hydrophobic absorption materials include, but are not limited to, natural, synthetic, and modified natural polymers. Three illustrative examples are cellulose fibers, which are coated with an hydrophobic compound, perfluoropolyether fibers and polypropylene fibers. Hydrophobic absorption materials are commercially available, for instance under the trade names K-Sorb Hydrophobic®, OCLANSORB, IRGASURF, and KLAUSORB.

For some applications it may be desired to provide an absorption layer that is at the same time both of a large capacity and designed to be easily penetrated. A large capacity may require a significant thickness, while an easy penetration may require thinner material. In some embodiments of the device the absorption layer is therefore of a non-uniform thickness. The absorption layer may for example posses one or more recessed regions at the surface opposing the sealing layer as illustrated in FIG. 2C. In other embodiments an additional guiding layer is located between the sealing layer and the absorption layer, as illustrated in FIGS. 4G and 4H. The guiding layer is easily penetrable, but does not provide sufficient absorption qualities. In other embodiments it does not provide any significant absorption qualities. The guiding layer thus serves two main functions. Firstly, it assists in transmitting fluid from the sample processing chamber to the absorption layer. Secondly, it prevents an undesired discharge, such as a sudden splash, into subsequent compartments of the device or into a subsequent device, when the sealing layer is penetrated. Therefore, the guiding layer assists the absorption layer in fulfilling its function of absorbing any desired fluid once the sealing layer is being penetrated. In some of these embodiments the absorption layer is designed to form a central voidage. In other embodiments the absorption layer consists of two or more separate parts as illustrated in FIG. 4G. In yet further embodiments the absorption layer consists of several parts of different properties (cf. FIG. 4H), such as for example fluid capacity, mechanical resistance to penetration, tensile strength or density. Thus in some embodiments the absorption layer includes parts that are easily penetrated and other parts that are of high absorption capacity. In some embodiments these parts form a macroscopic unity.

In other embodiments the absorption layer is made up of just one homogenous layer, which may be of uniform absorptivity. In any of these and the aforementioned embodiments, the absorption layer (e.g. a homogenous absorption layer) may be adapted to have sufficient capacity for absorbing a fluid of volume equating to the volume of the chamber. In other words, the absorption layer may be able to absorb a chamber-full of fluid.

The absorption layer contains in some embodiments fluidic means that direct any fluid, which is released from the sample processing chamber during penetration of the sealing layer, to a physical space. As an illustrative example, the absorption layer may contain or consist of a material that shows little absorptivity, but that is able to transfer fluid to a fluid line that direct it to a discharge unit. Examples of a respective fluid line include, but are not limited to, a channel or a tube. An embodiment of such an absorption layer is illustrated in FIG. 7B. In the depicted embodiment a tubing is able to discharge fluid that enters the absorption layer into a waste chamber. In some embodiments the absorption layer provides simply a space that is in fluid connection to a waste chamber. In these embodiments any splashing or discharging into subsequent compartments of the device or into a subsequent device (during penetration of the sealing layer) is prevented by a guiding layer and/or an additional supportive layer and/or an additional second absorption layer.

In a further embodiment, the entire device may be placed within a housing. The housing may also be adapted to take a plurality of said devices. Additionally, during the arrangement of a plurality of said devices, the housing may allow said plurality to be further secured together. Alternatively, the housing may be a raised platform or a means of further isolating the device in order to reduce and prevent the occurrence of contamination to samples during processing.

As mentioned above, the device may contain more than one sample processing chambers. In another embodiment, the device may thus include two, three, four or more sample processing chambers as for example illustrated in FIG. 4F or FIG. 7. The arrangement of a device having a plurality (meaning two or more) of chambers, allows to carry out several processes, e.g. chemical reactions, simultaneously or sequentially. Such a processing arrangement may allow for the combining of products at a later point in time. As an illustrative example, two reactants, which may be required for a subsequent process, can be activated or synthesized in a spatially segregated manner by using two separate sample processing chambers. As a further illustrative example, a plurality of chambers may be arranged in a geometric order, e.g. the form of a circle as shown in FIG. 7. Such a circular arrangement may allow a simultaneous processing of identical or different samples. Alternatively, matter may be processed sequentially in one or more chambers (See FIG. 16B).

All the above-mentioned embodiments of the device may be implemented in an apparatus for processing a biological and/or chemical sample. Accordingly, the present invention also refers to an apparatus for processing said samples. The apparatus includes at least one device, as described in any of the aforementioned embodiments, and a probe, wherein said probe is adapted to assist in the processing of the sample. As an illustrative example, the probe may selectively attract target matter from the sample processing chamber. Another illustrative of assisting in the processing of the sample example of regulating the temperature of fluid or the sample in the sample processing chamber. Typically, but not necessarily, the probe can be inserted into and removed from the sample processing chamber via the inlet of the device if required.

The probe may be designed to serve the function of attracting target matter through means such as physicochemical means or physical fields and forces such as for instance magnetic and electromagnetic fields or electrical charges. Three illustrative examples of attractions by physicochemical means are van-der-Waals interactions, ionic attractions and affinity attractions such as for instance antibody-antigen binding attractions. As described below, target matter may bind to the probe directly or via additional means, i.e. indirectly. Any part of the probe may be designed to attract certain target matter. As an illustrative example the probe may posses a head (head of probe), which, or a part of which, includes magnetic matter or provides a surface with affinity for certain target matter. FIG. 12 I depicts an embodiment, where a surface area of a head of a probe provides such a surface. Different areas of the same or several probes may also be designed to attract or “capture” the target matter. FIGS. 12 J and K depict two embodiments of a probe that possesses arms. Several areas of these arms are equipped with a different surface portion. Each of these surface portions is able to attract different target matter. Some of these areas may for instance be able to assist in antibody-antigen interactions, others apply charge attraction, others magnetic attraction and yet others oligonucleotide hybridisation (cf. below). The surface of the probe may also be adapted, through the incorporation of surface texturing or protrusions to provide recessed regions to or attract or capture the target matter. Further to “capturing” such matter, the recesses may also serve to protect the captured matter from being in direct contact with the sealing layer during the penetration process thereby resulting in the captured matter being brushed off from the probe. In addition, as mentioned above, in some embodiments the probe serves as a direct contact point with the sealing layer. Any part of the probe may be designed for such purpose.

FIGS. 14 and 15 illustrate embodiments of a probe, which is able to attract target matter and to shield it from any impact, for instance during the penetration of the sealing layer of the device. As described below, target matter may bind to the probe directly. As an illustrative example the probe may be equipped with affinity tags for cells or compounds. The target matter may also bind to the probe indirectly by using a substrate that is able to attract target matter. As an illustrative example, in some embodiments a medium for carrying out a chemical and/or biological process, which is filled into a sample processing chamber, comprises magnetically attractable particles. Such particles may be able to attract target matter. The magnetic beads can be functionalised with specific affinity for target matter and capturing target matter before it is being attracted to or released from the magnetic probe, therefore acting as an indirect binding means (see below).

Two illustrative examples of a probe depicted in FIGS. 14 and 15 include a head (head of probe). Inside this head magnetic matter may reversibly be placed. It is therefore able to attract magnetic particles which in turn are able to bind the target matter. In the embodiments shown, the head is located at the end of the primary core rod and serves as a binding region. The probes of the depicted embodiments also possess arms that can be adjusted to a protective position (FIG. 14A) with respect to any bound matter. In such cases, the bound matter on the head is shielded by these arms. The arms may be adjusted to a position in which bound matter is easily accessible or in which magnetic particles may be easily released from the binding region, i.e. in this case the head of the probe (FIGS. 14B and 14C). In other embodiments the probe may include a secondary core rod within a hollow primary core rod (sheath) that can be adjusted in its position. In some embodiments the secondary core rod is hollow.

In some embodiments binding and release of target matter to and from the probe is controlled through a change of the properties of the probe. An illustrative example is the change of magnetic properties of the probe as for instance illustrated above and in FIGS. 14B and 14C. In other embodiments the binding and release of target matter to and from the probe is controlled through a change in conditions of the sample processing chamber. Examples include, but are not limited to radiation, a change in temperature, a magnetic field, a pressure differential and the addition of matter into the sample processing chamber. As an illustrative example, the addition of matter may for instance affect the pH value of a solution comprised in the sample processing chamber. Where it is not desired to control the binding of target matter to the probe, a singular binding process may occur once the probe is inserted into the device of the invention and target matter may remain bound to the probe throughout the remaining processes performed within the device of the invention.

An attraction or binding of target matter to the probe may be achieved by any means. It may in some embodiments be desired to select means that do not affect, change, spoil or damage the target matter. In other embodiments the means of attracting or binding target matter may be selected to be part of a desired process to be performed in the device of the invention.

In one exemplary embodiment, the probe contains a substrate capable of binding target matter such as, but is not limited to, DNA, RNA, a synthetic analogue thereof, a protein, bacterial cells or spores, viruses, or low molecular weight organic molecules. The substrate thus has a higher affinity to the target matter than to other matter. The probe may for instance be coated with a respective substrate. Examples of a respective substrate include, but are not limited to, an antibody, a fragment thereof and a proteinaceous binding molecule with antibody-like functions. Examples of (recombinant) antibody fragments are F_{ab} fragments, F_{V} fragments, single-chain F_{V} fragments (scF_{V}), diabodies or domain antibodies (Holt L J et al., Trends Biotechnol. 21(11), 2003, 484-490). An example of a proteinaceous binding molecule with antibody-like functions is a mutein based on a polypeptide of the lipocalin family (WO 03/029462, Beste et al., Proc. Natl. Acad. Sci. USA 96, 1999, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see WO 96/23879), proteins based on the ankyrin scaffold (Hryniewicz-Jankowska A et al., Folia Histochem. Cytobiol. 40, 2002, 239-249) or crystalline scaffold (WO 01/04144,) and the proteins described in Skerra, J Mol. Recognit. 13, 2000, 167-187. In addition the probe may be coated with a modifying agent that further increases the affinity of the substrate for any or a certain form, class etc. of target matter.

In another exemplary embodiment, the probe is able to attract a substrate. The substrate in turn is capable of attracting or binding target matter. Magnetically attractable particles (functionalized or otherwise) used to capture the target matter are an illustrative example of such a substrate. In such an embodiment, the probe design may provide a hollow primary core rod (sheath) (cf. e.g. sheath 25 in FIGS. 12B and 12H), which due to the hollow design, allows for the integration of a secondary core rod with magnetic end extended to the head of the probe (cf. e.g. secondary core rod 16 in FIG. 12B) for use of the purpose, not limiting, to binding magnetic particles, as illustrated in FIG. 12B or 12H. Such secondary a core rod may itself be able to contain fluid through the presence of channels within the sheath or secondary core rod. The channels may furthermore extend into the head of the probe, as illustrated in FIGS. 12B and 12H. Alternatively the core rod may contain a similar binding end, for instance integrated therein (cf. e.g. FIG. 12D or FIG. 12E).

For convenience magnetically attractable particles are herein referred to as “magnetic particles” or “magnetic beads”. Magnetic particles may contain ferromagnetic or supermagnetic material. Supermagnetic material responds to a magnetic field with an induced magnetic field without a resulting permanent magnetization. Magnetic particles based on iron oxide are for example commercially available as Dynabeads® from Dynal Biotech, as magnetic MicroBeads from Miltenyi Biotec, as magnetic porous glass beads from CPG Inc., as well as from various other sources such as Roche Applied Science, BIOCLON, BioSource International Inc. or Novagen Inc., to name a few. Magnetic nanoparticles based on supermagnetic Co and FeCo, as well as ferromagnetic Co nanocrystals have been described, for example by Hütten, A et al. (J. Biotech. (2004), 112, 47-63).

As mentioned above, an alternative embodiment may allow a secondary core rod to be inserted into a sheath (hollow primary core rod) to form the probe (cf. e.g. core rod 16 in FIGS. 12-15). The sheath may be adapted to serve in shielding the core rod from a fluid or chemical compound in the cavity. The sheath typically extends into the head of the probe, which, in one embodiment may be made up of either an integrated permanent magnet or an integrated electromagnet. Alternatively, embodiments having a secondary core rod, may allow for the movement of said secondary core rod, within the sheath, in a longitudinal direction. This results in the magnetic end of the secondary core rod being capable of lifting above the fluid level, in case where the magnetic beads are dispersed, or being lowered (fully submerged) below the fluid level when magnetic beads are to be attracted or concentrated onto the probe head.

In other embodiments the probe may simply consist of a long rod ending in a head (which may serve in binding target matter) as depicted in FIG. 12 A. In further embodiments the probe includes a solid primary core rod, a head and a plurality (two or more) of arms, which may be arranged as surrounding the primary core rod. The head of the probe, including the core rod, may function in binding target matter along with the end of the arms, which may be coplanar or otherwise with respect to the head of the primary core rod. In an exemplary embodiment, the primary core rod (or secondary core rod, if present) directly provides a surface for attachment of target matter, and the arms merely aid in agitating the fluid. The core rod of the probe may allow for the use of different classes of capturing surfaces to capture different classes of target matter. Examples of capturing surfaces include, but are not limited to, aluminum trioxides and silicon dioxides, thiolated surfaces, aminated surfaces and silanated surfaces.

The core rod and any arms, blades etc. attached thereto may also serve the function of penetrating the penetrable sealing layer (and absorption layer, if necessary) to form the outlet for the fluid to depart the chamber. Accordingly, the departing fluid is absorbed by the absorption layer in fluid contact with said sealing layer. In embodiments of the device where at least two devices are in fluid connection with each other (either via stacking or horizontal arrangement), the probe, with the attached sample substance, may be used to transfer said matter from one chamber to another for further processing without any exposure to the environment.

In a further embodiment, the arms of the probe may be adapted to serve as a protection for a part of the probe. As an illustrative example arms of the probe may protect a head of the probe to prevent target matter attached thereto from brushing off by the broken edges of the penetrated sealing layer. In such an embodiment, the ends of the arms may extend beyond the head of the probe.

In some embodiments, the arms may have single or multiple channel(s) embedded in them. In one such embodiment the channels are in fluid connection with external fluid reservoirs from the environment. Accordingly, said channels may be used for dispensing fluids into the chamber of the device, which is otherwise sealed of from the external environment. The connection to the external environment may be pressurized thereby enabling the probe to also have a suction and dispensation means to enable it to mix, stir etc. any fluid and other matter within the chamber to ensure a uniform mixture where needed. In such an embodiment, the probe may be also adapted to introduce and remove the test sample into the sample processing chamber thereby reducing the risk of contamination, as the probe would remain within the chamber from the very beginning.

The channels may also be used for dispensing sealants into the chamber of the device. The sealant may be room temperature curing polymers, heat curing polymers, or photo-curable polymers, for example. In this regard, the sealant may be dispensed at any stage of the processing sequence but it may be preferably carried out in the last stage. As an illustrative example, in the extraction of nucleic acids, the last step is the elution step. In said elution step, a silicon sealant is dispensed on top of the collecting tube or recess wherein the target matter was located. The purpose of dispensing a sealing layer is a reduction of the risk of exposing a sample to contamination while collecting it. As an illustrative example, an arrangement as depicted in FIG. 11A may be used to collect a sample.

The probe may possess arms of the dimensions shown in e.g. FIG. 11A or 11B, however containing channels as for instance depicted in FIG. 12G, FIG. 12I and FIG. 13D. In this embodiment the openings of the channels end in direct proximity to the sealing layer of the device, while the probe is located in a position where its head is located in a medium that may contain target matter. When a sealant is dispensed through the opening of the channels, it may secure the head of the probe in its position. Where the arms form (e.g. bell-shaped) continuous unity, dispensing a sealant through respective channels may furthermore hermetically seal the area within the arms of the probe from the remaining sample processing chamber. The probe having dispensing channels as described above, may also dispense other reagents as required by the reaction process being carried out.

In a further embodiment, arms, blades or other elements surrounding the core rod, such as a hollow cylindrical structure for example, serve as a means of protection for matter that is attached to the probe, as illustrated in FIGS. 14 and 15. The elements such as arms or blades may be mechanically adapted to perform vertical movements of a certain distance independent of the core rod, i.e. without the head of the probe moving from their present position. In allowing such vertical movements, the arms or blades may move from a first vertical position, wherein the arms or blades are designed to function as a protector during the penetration of the probe to the next chamber. After the successful penetration, the arms or blades may traverse to a second vertical position, wherein this second vertical position allows for the effective dispersion of target matter to the reagents inside the chamber.

The probe is in some embodiments adaptable to various embodiments of the devices, such as different dimensions of the chamber, different sizes of recesses of the sealing layer etc. As an illustrative example, a probe may possess diametrically translatable arms. Such a probe is thus in some embodiments able to alter the distance between a central head and the translatable arms. Such a probe may furthermore be able to alter its diameter relative to the sealing layer. This may for instance result in a variable penetration area in the sealing layer, which allows the invention to work for different volumes in each chamber. In this regard, the invention may be suitable for use in mixing very small volumes, such as for instance volumes in the range from 5 μl to 10 μl, effectively.

In other embodiments, the probe or a part of the probe, with a sample bound to it, may be mechanically adapted to perform vertical movements, as described above. As an illustrative example, the head of the probe may be able to move vertically independently from the outer sheath and the arms. The head may, by vertically travelling a certain distance, expose matter, for instance a sample, to a fluid or a specialised compartment present in the chamber. An exposure to a fluid may result in the formation of a suspension or a solution. As a consequence a reaction with reagents present inside the chamber (e.g. dissolved in the fluid) may occur. Where a specialised compartment of the chamber contains reagents, a reaction may likewise occur. After a respective reaction has reached a desired degree of completeness, matter such as a sample may be recaptured onto a surface of the probe, for instance again to the head of the probe. Thereafter the head may retract vertically again until it re-enters the sheath of the probe.

In some embodiments of the apparatus, the probe is furthermore able to create the outlet of the chamber by penetrating the sealing layer. The probe may achieve this by having one or more pointed tips or sharpened edges, or alternatively, the probe may include a screw-like design such as that of a thread by which it may “drill” through the penetrable sealing layer.

In other embodiments the probe itself need not provide any particular design that assists the penetration of the sealing layer. In such embodiments the probe may posses an end of any design. Examples include, but are not limited to a flat end, a rounded end or a chiseled end. In such embodiments the penetration of the sealing layer may be achieved by means other than the probe, such as pressure, mechanical means, a change in temperature, and a change in pH (see above). In other embodiments the penetration of the sealing layer may be achieved by pure force, such as, but not limited to, mechanical force. In some of these embodiments the sealing layer includes a design that assists a penetration, for example by a probe, as described above.

Any part of the probe may be designed to penetrate the sealing layer of the device. As an illustrative example, in some embodiments a central rod of the probe ends in a head that juts other parts of the probe and will thus penetrate a sealing layer, especially where the sealing layer is of flat design (cf. e.g. FIG. 11G). In other embodiments a respective head of the probe branches into several protruding ends that jut other parts of the probe. As a further illustrative example, in yet other embodiments the probe includes blades or arms that jut other parts of the probe (cf. e.g. FIG. 11B) and thus penetrate the sealing layer. It will be understood that also the design of the sealing layer determines, which part of the probe penetrates the sealing layer first. In embodiments where the sealing layer is of convex design (cf. e.g. FIG. 11F), a probe as e.g. depicted in FIGS. 11B, 11F or 11H may penetrate the sealing layer with its head. In embodiments where the sealing layer is of concave design (cf e.g. FIG. 11E), a probe as depicted in e.g. FIG. 11E or 11G may penetrate the sealing layer with its arms.

In other embodiments, the probe is adapted to agitate a fluid (or medium) contained within the chamber. The agitation may be in the form of a vertical, horizontal, rotational or a combinatorial displacement thereof of the fluid within the chamber. In order to achieve such a displacement, the probe may be adapted to have an impeller, vanes, a propeller or arms mounted on the portion of the probe within the chamber.

In further embodiments, the probe may have magnetic properties. Some embodiments of a magnetic probe are used in conjunction with embodiments of the device having magnetic particles contained within the chamber. In some of these embodiments matter such as reagents is coated onto suspended magnetic particles in the fluid. The magnetic properties may be induced through an electromagnet or by using permanent magnets integrated into the probe, as previously mentioned.

In yet another embodiment, the motion (rotation and penetration) of the probe may be mechanized. In such a mechanized setup, a single device or multiple devices may be arranged in series or in parallel with each other. The arrangements allow for single or multiple processes to be conducted simultaneously and/or sequentially across the different devices. In the above embodiment in which the device is incorporated into a biomedical device, such mechanization may also be implemented therein.

In a further embodiment, the device or the apparatus may include an elution chamber (cf. e.g. FIGS. 8 and 9). The elution chamber may be adapted to receive a head of the probe. In some embodiments the elution chamber is adapted to be detachable from the apparatus. Alternatively, the elution chamber may be in further connection with another device for additional analysis, such as, but not limited to, a cytometer, a spectrometer, a colorimeter and a real-time thermal cycler, for example. In some embodiments the elution chamber is of smaller volume than the sample reaction chamber of the device.

In other embodiments, multiple arms or blades etc. are arranged with respect to the elution chamber such that during the elution step, only the head of the probe, but not the arms, is inserted into the elution chamber. As an illustrative example, the probe depicted in FIG. 9 may have penetrated the lowest sealing layer of the depicted device. Where it is in further downward motion, the probe will be halted once the arms contact the ground of the lowest compartment. In this position the head of the probe, which is located between the arms at the end of the core rod (cf. FIG. 12B), is inserted into the elution chamber depicted in FIG. 9. A respective elution chamber may be selected of any desired volume, including, but not limited to, volumes in the order of magnitude below 100 μl. Hence, such a design enables the achievement of small volume elution, which in turn facilitates the concentration of target matter.

In another embodiment, a magnetic field can be applied by positioning one or more magnetic bars or magnetic rods externally and/or internally in the device in near proximity to the probe to speed up the release magnetic particles from the probe during the dispersing process. The magnet field can be activated and deactivated between a collecting position and a releasing position. As an illustrative example, a spacer as depicted in FIG. 6D may be replaced by a magnet to attract magnetic particles downward or upward, while the magnetic particles are located in an elution chamber or a sample processing chamber adjacent to such spacer respectively.

The device of the invention may be used for any analytical or preparative purposes or combinations thereof. An illustrative example of an analytical application is determining the effect of chemoattractants on a microorganism. Methods that determine such effects are usually referred to as a “cell migration assay”. For carrying out such a method the probe may include a respective chemoattractant, which may optionally be covered by a membrane of a defined pore size. Such a membrane may for instance be coated with components (e.g. fibronectin) that provide assistance for a microorganism in adhering to the probe. Where the respective microorganism adheres to the probe, it may, after a defined period of time, be further processed as generically illustrated below. The microorganism may be analysed while adhering to the probe or after a release. Methods of releasing cells from a substrate are well known in the art and include for instance the use of calcium-chelating compounds or the application of force, for example centrifugation.

The device is capable of processing matter by the performance of any desired method. Examples include, but are not limited to, a chemical reaction, a component exchange, disintegration, and disengagement and any combination thereof. An illustrative example of a disintegration is a cell lysis. Two illustrative examples of a disengagement are an extraction of a molecule from an organism or a part of an organism and a release of a molecule from an organism. An illustrative example of exchanging components of a sample is a washing process. At the beginning and at the end of a washing process target matter or a precursor thereof, which may be comprised in the sample, is typically bound (whether directly or indirectly via for example magnetic beads, see above) to the probe. The transfer of target matter into a fluid, its release into the fluid, its mixing with the fluid (for instance by agitation), and its subsequent recovery, make up exemplary steps of a washing process.

In some embodiments the target matter is a molecule that is suspected or known to be present within other (undesired) matter, from which it needs to be extracted. Extraction of a molecule from an organism or a part of an organism may for instance include the usage of a compound that facilitates the transfer of a desired molecule from an organism or a part thereof into a fluid. An illustrative example of an extraction of a molecule from a part of an organism is an extraction of proteins (wholly or partly) integrated into the cell membrane. It is often desired to transfer such proteins into an aqueous solution for further processing. A compound that facilitates the transfer of such proteins into an aqueous solution is a detergent. Contacting a respective cell membrane with an aqueous solution, to which a detergent is added, will typically result in an extraction of membrane proteins.

Any chemical reaction may be performed within a device of the present invention, as long as the device remains essentially intact while the reaction is performed. For some chemical reactions it may thus be required to select (a) material(s) for the surfaces of the device that possesses a particular resistance to stress occurring during the reaction, such as for instance heat or corrosive properties of the target matter, reactants, products or intermediate products. Examples include, but are not limited to, a protein synthesis, a nucleic acid synthesis, a peptide synthesis, and enzymatic degradation, an interaction with a binding molecule or any combination thereof.

The sample may be of any origin. It may for instance, but not only, be derived from human or non-human animals, plants, bacteria, viruses, spores, fungi, or protozoa. Accordingly, any of the following samples selected from, but not limited to, the group consisting of milk soil, a blood sample, a serum sample, a plasma sample, a urine sample, a semen sample, a lymphatic fluid sample, a cerebrospinal fluid sample, a milk sample, an amniotic fluid sample, a biopsy sample, a needle aspiration biopsy sample, a cancer sample, a tumour sample, a tissue sample, a cell sample, a cell lysate sample, a virus culture sample, a crude cell lysate sample, a forensic sample, an archaeological sample, an infection sample, a nosocomial infection sample, a production sample, a drug preparation sample, a biological molecule production sample, a protein preparation sample, a lipid preparation sample, a carbohydrate preparation sample, or any combination thereof may be processed by the device. Where desired, a respective sample may have been preprocessed to any degree. As an illustrative example, a tissue sample may have been digested, homogenised or centrifuges prior to being used with the device of the present invention. The sample may furthermore have been prepared in form of a fluid such as a solution. Examples include, but are not limited to, a solution or a slurry of a nucleotide, a polynucleotide, a nucleic acid, a peptide, a polypeptide, an amino acid, a protein, a synthetic polymer, a biochemical composition, an organic chemical composition, an inorganic chemical composition, a lipid, a carbohydrate, a combinatory chemistry product, a drug candidate molecule, a drug molecule, a drug metabolite or of any combinations thereof. Further examples include, but are not limited to a suspension of a cell, a virus, a microorganism or of any combinations thereof. It is understood that a sample may furthermore include any combination of the aforementioned examples.

Often, but not necessarily, the sample will include, or will be expected to include, the target matter or a precursor thereof. Such embodiments shall be illustrated by a number of examples: The target matter may for instance be a cell or a molecule added to or comprised in the sample, and it may be desired to obtain it in a purified or enriched form. As another example, the target matter may be a compound known or theorized to be obtainable from a precursor compound by means of a chemical process. In this case the sample may for instance include a solution of such precursor compound. As yet a further example, it may be desired to remove a contaminant from a fluid, for instance from a biological sample. In this case the device may be used for decontaminating or analysing a sample and the contaminating matter is understood as the target matter. As an illustrative example, a cell culture media may be suspected to be contaminated. In this case it may be desired to identify the type of contaminant and to use the device of the invention for this purpose. The probe possesses in such embodiments an affinity to the contaminant or it is able to bind other matter that has an affinity to the contaminant.

The target matter or precursor thereof may thus be of any nature. Examples thus include, but are not limited to, a nucleotide, an oligonucleotide, a polynucleotide, a nucleic acid, a peptide, a polypeptide, an amino acid, a protein, a synthetic polymer, a biochemical composition, an organic chemical composition, an inorganic chemical composition, a lipid, a carbohydrate, a combinatory chemistry product, a drug candidate molecule, a drug molecule, a drug metabolite, a cell, a virus, a microorganism or any combinations thereof. In embodiments where the target matter is for example a protein, a polypeptide, a peptide, a nucleic acid, a polynucleotide or an oligonucleotide, it may contain an affinity tag. Examples of affinity tags include, but are not limited to biotin, dinitrophenol or digoxigenin. Where the target matter is a protein, a polypeptide, or a peptide, further examples of an affinity tag include, but are not limited to, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala and the “myc” epitope of the transcription factor c-myc of the sequence Glu-Gln-Lys-Leu-Ile-Ser-Glu-Glu-Asp-Leu. Where the target matter is a nucleic acid, a polynucleotide or an oligonucleotide, an affinity tag may furthermore be an oligonucleotide tag. Such an oligonucleotide tag may for instance be used to hybridize to an immobilized oligonucleotide with a complementary sequence. A respective affinity tag may be located within or attached to any part of the target matter. As an illustrative example, it may be operably fused to the amino terminus or to the carboxy terminus of any of the aforementioned exemplary proteins.

The device of the invention may be used manually, automatically or in a combination thereof. An automatic use may include a control system that induces in a programmed manner the performance of any acts desired for a process to be carried out using the device of the invention. Examples include, but are not limited to, motion of a probe into or within the device, pumping, injecting, stirring, the application of energy and the start of a detection machine. The use may be an integrated step of, for instance, a method of processing (e.g. isolating, reacting etc.) one or more samples. Such a method may include a number of sequential steps. As an illustrative example, the device may be used for the isolation of a desired cell population from a blood or tissue sample. An initial step of a method of processing a respective sample may be aimed at a volume reduction and include for example centrifugation. A late step of a method of processing a respective sample may include the detection of desired cells, for instance by means of an automated cell counting device. As a further illustrative example, the device may be used to perform a chemical reaction. Where the reaction product is a liquid of suitable boiling point, a late step of its manufacture may include a distillation process. For this purpose an obtained reaction mixture may be heated. The use of the device may also form a complete process, all steps of which are carried out within the device of the invention.

Thus the device is in some embodiments for instance an external, internal or integrated part of a larger apparatus. A respective larger apparatus may include any desired component. It may for example include one or more components that serve in keeping any element of the device of the invention in a desired position or one or more components that assist or serve in moving any element of the device of the invention into such a desired position. An example of such a larger apparatus that includes the device of the present invention is shown in FIG. 18. The depicted embodiment includes a motor that controls a probe, which can be inserted into the device of the invention. It furthermore includes means such as screws and holders to fix the position of the device of the invention as well as the probe. Further elements of the depicted embodiment include means for temperature regulation such as a hotplate and a temperature controller 48. In other embodiments the device includes further components adapted to perform any function desired to be performed. An illustrative example of a component that may be part of a respective apparatus or the device itself is a blender serving as a tissue homogenizer. A further illustrative example of a component that may be part of a respective apparatus or of the device is a spectrometer such as a fluorescence spectrometer, which serves in detecting a processed sample obtained by using the device, such as a cell or a compound.

In this respect the present invention also provides a method for processing a biological and/or chemical sample using the above described device, the method comprising the following steps:
(a) Providing a device for processing a biological and/or chemical sample as described above;
(b) Providing a biological and/or chemical sample;
(c) Placing the sample a into the sample processing chamber of the device, thereby forming a reaction mixture; and
(d) Exposing said reaction mixture to a process, thereby forming a product mixture.

In some embodiments, the method further includes in step (b) providing target matter or a precursor thereof, as well as in step (c) placing the target matter or precursor thereof into the sample processing chamber of the device. As already indicated above, in other embodiments the target matter is included in the biological and/or chemical sample. In some embodiments, the method further includes:
(e) Selectively attracting target matter suspected to be comprised in the product mixture to the surface of a probe; and
(f) Using said target matter, with or without release from the probe, for further use such as for storage, detection, analysis and disposal.

In an exemplary illustration of the use of the apparatus, a reaction involving magnetic particles may be used, as mentioned above, in the isolation of nucleic acids, for example. As a further illustrative example, the apparatus may be used for an organic compound to undergo a chemical reaction (such as e.g. reduction or oxidation). A product obtained by such chemical reaction may be of different hydrophilic properties when compared to the compound used as a starting material. As an illustrative example, a starting material or a reactant used may be too hydrophilic to bind to a surface of for instance a probe or beads possessing a hydrophobic surface. The reaction product may however be hydrophobic enough to be attracted to such a surface. Particles equipped with a suitable hydrophobic surface may thus bind a respective reaction product without binding starting material or a reactant. The particles may further be equipped with a magnetic core, so that they can be used to recover such a reaction product. In some embodiments, the magnetic particles are coated with a substance that has an affinity for nucleic acids or organic compounds. Accordingly, when agitated by the probe using any one of the above-mentioned agitation methods, a uniform distribution of magnetic particle is achieved throughout the chamber. The magnetic particles bind to the desired nucleic acid or reaction product and remain distributed throughout the chamber.

Subsequently, the insertion of the magnetic probe into the chamber creates a magnetic field, which attracts and holds the magnetic particles (and nucleic acids or reaction products bound thereto) to the probe. Consequently, a large proportion of the desired nucleic acid or reaction product can been removed from the medium (test sample) and the remaining medium is no longer essential. Hence, with the probe finally creating an outlet in the penetrable sealing layer, the unwanted medium may be released via the outlet and to the absorption layer to be absorbed.

Should there be a need to further process a sample of the extracted nucleic acid or recovered reaction product, such as washing, for example, the device, as mentioned above, may be arranged such that the outlet is in fluid communication with the inlet of another device. Such an arrangement may be achieved if several devices are collinearly stacked, for example. Accordingly, the probe of the above illustrative example may be further inserted through the outlet of the sealing layer, past the absorption layer and into the chamber of the subsequent device to further process the extracted nucleic acid as required.

As described above, target matter may be attracted or collected, for example to a probe inserted via the inlet of the device. Typically the method of the invention also includes a penetration of a sealing layer of the device (cf. e.g. FIG. 10 or FIG. 16). Any suitable means may be used to penetrate the sealing layer (cf. above for examples). In some embodiments of the method of the invention the sealing layer of the device is penetrated by mechanical force, exercised by the probe. Subsequently the absorption layer may soak up, incorporate, suck in, transmit or otherwise absorb or accept a certain amount of fluid released from the sample reaction chamber, via the outlet that is created upon penetration of the sealing layer. The penetration of the sealing layer may be performed at any point in time with respect to an attraction of target matter. In some embodiments the sealing layer is penetrated after target matter suspected to be comprised in the product mixture has been attracted to the surface of the probe. In other embodiments both the attraction of target matter and the penetration of the sealing layer occur simultaneously. In yet other embodiments, the sealing layer is penetrated first and target matter attracted or collected subsequently.

Further processing may include, but is not limited to washing, purification and removal of a product from magnetic particles, which is derived by means of a process carried out in the device, for example, a nucleic acid or the product of a chemical reaction. As an illustrative example, an extracted nucleic acid may be released from the probe or any capturing surfaces at the end of the process, into an elution chamber. The elution chamber may be provided as part of the apparatus.

In some embodiments it may be desired to further react a resulting organic compound, which is the product of a chemical reaction performed in a device of the invention. The chamber of the subsequent device may thus be used to perform such a further reaction process. Thus a series of steps of an organic synthesis may be performed using an arrangement of several devices of the invention.

As indicated above, the chamber of the device may be filled with a medium for carrying out a chemical or biological process. Examples of the media that may be used include, but are not limited to, organic solvents and buffer solutions. Examples of buffer solutions include, but are not limited to, lysis buffers, binding buffers and washing buffers such as phosphate buffered saline (PBS), for example.

Numerous organic solvents are used in the art and can be used to carry out the various processes described herein. Examples of solvents include, but are not limited to, hexane, heptane, cyclohexane, benzene, toluene, p-xylene, pyridine, dichloromethane, chloroform, carbon tetrachloride, carbon disulfide, tetrahydrofuran, dioxane, diethyl ether, diisopropylether, ethylene glycol monobutyl ether, tetrahydrofuran, methyl ethyl ketone, methyl isobutyl ketone, acetone, cyclohexanone, ethyl acetate, isobutyl isobutyrate, ethylene glycol diacetate, dimethylformamide, acetonitrile, N,N-dimethyl acetamide, nitromethane, acetonitrile, N-formylpiperidine, N-methylpyrrolidone, dimethylsulfoxide, methanol, ethanol, butyl alcohol, formic acid, dimethylarsinic acid [(CH₃)₂AsO(OH)], N,N-dimethyl-formamide, N,N-diisopropylethylamine, chlorophenol, acetic acid, tert.-butyl alcohol, phenol, cyclohexanol, and aniline, to name a few.

Numerous buffer compounds are used in the art and may be used to carry out the various processes described herein. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroxyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[Tris(hydroxymethyl)-methylamino]-1-ethansulfonic acid (also called TES), 2-cyclohexylamino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)aminomethane (also called TRIS), bis-(2-hydroxyethyl)-imino-tris(hydroxymethyl)methane (also called BIS-TRIS), and N-[Tris(hydroxymethyl)-methyl]-glycine (also called TRICINE), to name a few. The buffers may be aqueous solutions of such buffer compounds or solutions in a suitable polar organic solvent.

In addition to the above, the medium may also contain additives, such as, but not limited to, reagents, catalysts and reactants, for carrying out a chemical or biological process. As an illustrative example, salts, substrates or detergents may be added in order to maintain cells or proteins in an intact state. As a further illustrative example, chelating compounds may be required, for instance to protect organisms from traces of otherwise toxic salts or to increase the yield of a chemical reaction. As yet a further illustrative example, protease inhibitors may need to be added in order to maintain proteins in an intact state. A further example of a possible additive to a medium includes magnetically attractable particles (see above).

As an illustrative, but not limiting example, the device of the present invention may be used to carry out a sandwich-type enzyme-linked immunosorbent (ELISA) assay. The uses and capabilities of this assay are well known to those skilled in the art. By combining several devices, wherein each device contains at least one or more of the requisite reagents for the binding, washing and detection steps, it is possible to assay for the presence of target matter in a sample. As described above, the capture reagent may be directly or indirectly coupled to the probe. In a specific embodiment, the capture reagent is an antibody targeted to an antigen of interest. More specifically, the antibody may be directed to an antigen present in a HIV virus and the sample blood or serum suspected to contain this HIV virus. Detection of an antigen can be monitored through colorimetric and or spectroscopic analysis. This can occur after elution of substrates captured onto the probe or within the device itself without recourse to an elution step.

The following drawings, accompanying descriptions thereof, and the subsequent examples serve in illustrating the invention and to further aid in the clarity and understanding of the invention. However, it should be noted that the scope of the invention is by no means limited to the various embodiments illustrated hereafter.

### BRIEF DESCRIPTION OF THE DRAWINGS

FIG. 1 compares the method and device of the present invention to a standard method and device of the prior art.

FIG. 2 depicts exemplary embodiments of a device of the invention, containing a sample processing chamber 2 with an inlet 7, a sealing layer 9, and an absorption layer 3.

FIG. 3 shows exemplary embodiments of the sealing layer.

FIG. 4 depicts further exemplary embodiments of a device of the invention containing additional elements, such as an additional supportive layer 5 or a guiding layer 11.

FIG. 5 shows examples how several devices of the invention may be arranged.

FIG. 6 shows exemplary embodiments of one device, which includes several sample processing chambers 2, sealing layers 9 and absorption layers 23.

FIG. 7 shows embodiments how elements of one device may be arranged as well as further examples of how several devices of the invention may be arranged.

FIG. 8 depicts an exemplary embodiment of an apparatus that contains several devices of the invention as well as a probe 13. The depicted embodiment contains furthermore an elution chamber 12.

FIG. 9 depicts an exemplary embodiment of a method of the present invention. Three stages of the method are shown.

A close-up view of these depicted stages, focussing on the movement of the probe, is shown in FIG. 10.

FIG. 11 depicts exemplary embodiments of a sample processing chamber 2 filled with fluid. Most of the depicted embodiments further include a recess 29 in the sealing layer 36. The recess is able to accommodate a very small amount of a fluid 10.

FIG. 12 depicts exemplary embodiments of probe 13.

A close-up view of some of these embodiments is shown in FIG. 13.

FIGS. 14 and 15 depict two exemplary embodiments of means of shielding and releasing of magnetic beads 4 from a probe 13.

FIG. 16 shows exemplary embodiments of a method of the present invention.

FIG. 17 shows the amplification analysis in a PCR machine of RNA extracted using the method of the invention.

FIG. 18 depicts an example of a configuration of an apparatus of the present invention.

### Exemplary Embodiments of the Invention

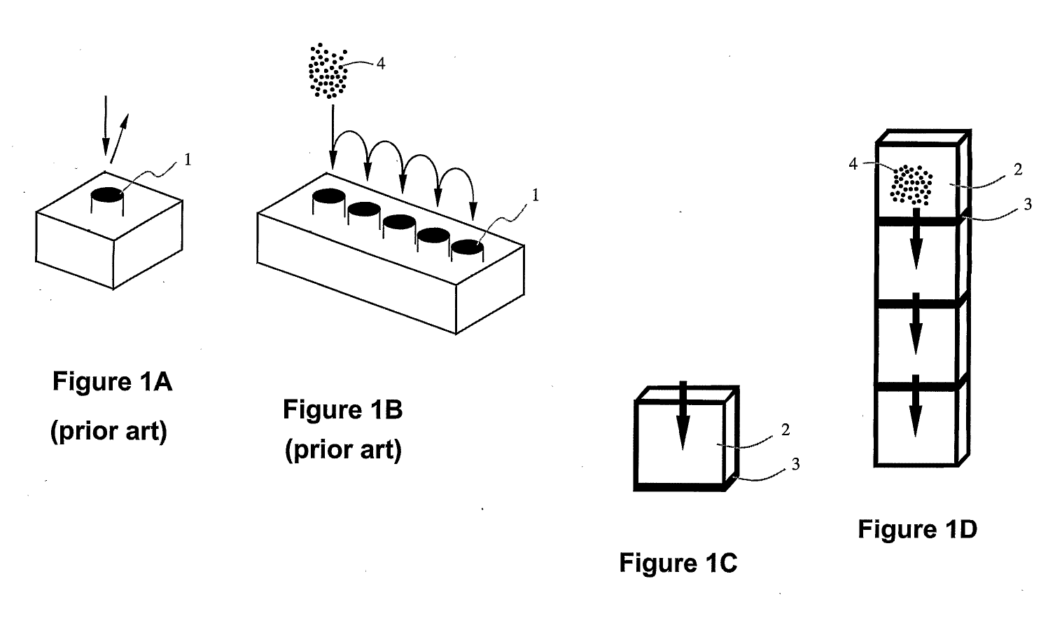

FIG. 1 compares the method and device of the present invention to a standard method and device of the prior art.

In a standard device for processing a sample, a sample is introduced into a container 1 (FIG. 1A). Typically it is introduced in form of a solution. It is reacted or analyzed with the help of reactants or reagents. Subsequently or during the reaction, the solution may need to be removed. Alternatively sample molecules may be recovered from the container by means of magnetic beads 4. In cases where a process involves several analytic and preparative steps it may be required to expose the sample molecules to a different solvent, buffer etc. or to otherwise different conditions. This requires either the removal of the magnetic beads together with any bound molecules, to another device or a removal of the solution in the container for further processing. Currently used standard devices generally allow only for the transfer of the magnetic beads to a different device as shown in FIG. 1B. Typically this requires that the previous device be opened, if it was sealed for carrying out a reaction, an analytical or a washing step. Each time sample molecules are transferred by means of magnetic beads there is thus a risk of contamination.

In contrast to this prior art, the device of the present invention does not require removing the sample from a container. As shown in FIG. 1C, the device of the present invention allows for the removal of a fluid from a sample processing chamber 2 without the need to open the respective chamber. The sample may be of any state such as for instance a chemical, organic, biological, solid, liquid, emulsion, suspension, colloidal, or composite material. The sample forms a mixture together with any reagent or chemical already present or subsequently added to the device. In some embodiments sample molecules, analytes etc. may be bound to magnetic beads or other capturing substrates. The device of the invention allows retaining such beads and at the same time disposing a fluid from the sample processing chamber 2 by absorption into an absorption layer 3.

As schematically depicted in FIG. 1D, where several analytic or preparative steps are to be performed, magnetic beads, with any target matter attached thereto, may thus be transferred from one sample processing chamber to another without the need to expose them to the ambience. A risk of contamination by opening a container is thus omitted.

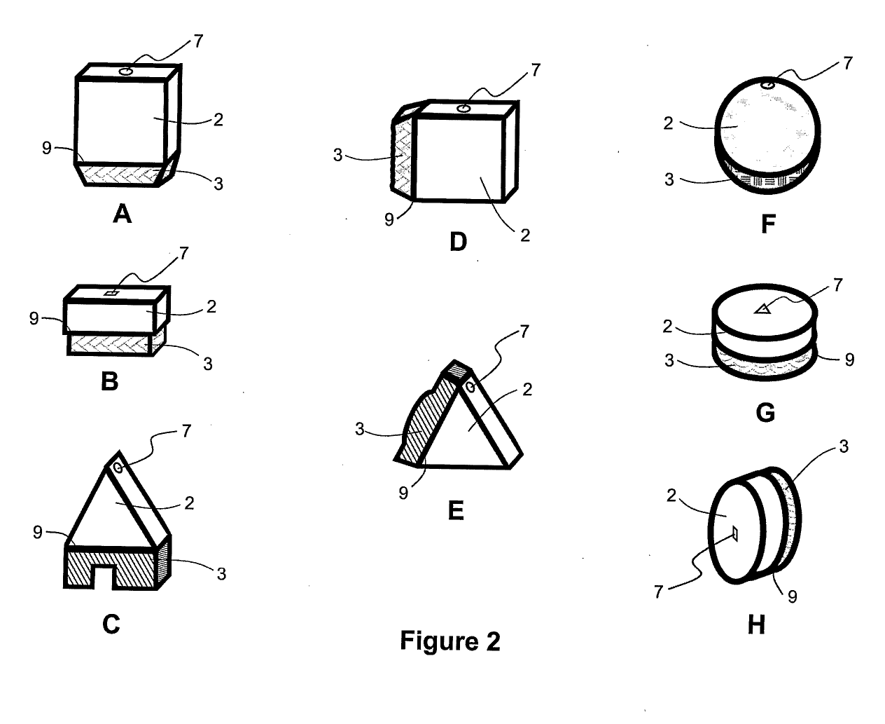

FIG. 2 depicts exemplary embodiments of a device that contains a sample processing chamber 2 with an inlet 7 at one end and a penetrable sealing layer 9 at a second end. The sealing layer 9 forms a part of an inner wall of chamber 2. In embodiments shown in FIGS. 2A, B, F, C, G and H, inlet 7 and the sealing layer coaxially oppose each other. Within this group of embodiments, FIGS. 2A, B, F, and G show a device, in which the inlet faces the exact center of the sealing layer. In the embodiments depicted in FIGS. 2C and H inlet 7 opposes a part of the sealing layer which differs from its exact centre. FIG. 2D shows an embodiment where inlet 7 is positioned orthogonally to the sealing layer. FIG. 2E shows an embodiment where inlet 7 is adjacent to the sealing layer. It should be understood that elements of the device, such as for example layers, need not be of uniform thickness, length, depth, height etc. As depicted in FIG. 2, the device further includes an absorption layer 3, which is in fluid contact with sealing layer 9 upon penetration of the sealing layer. The embodiments depicted in FIGS. 2C and 2E posses an absorption layer with a recess and a bulge respectively.

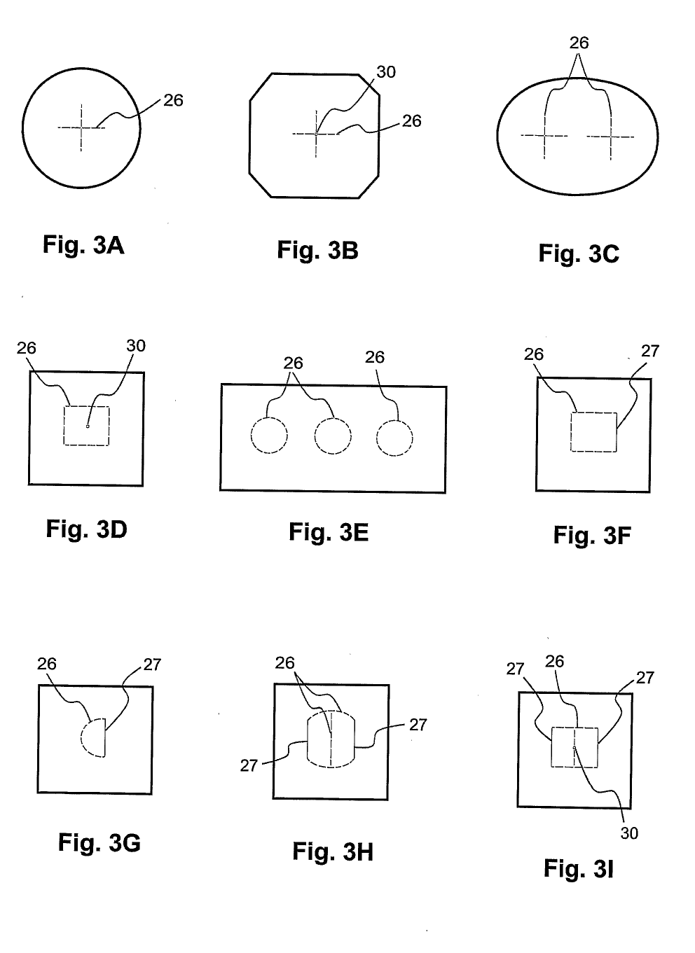

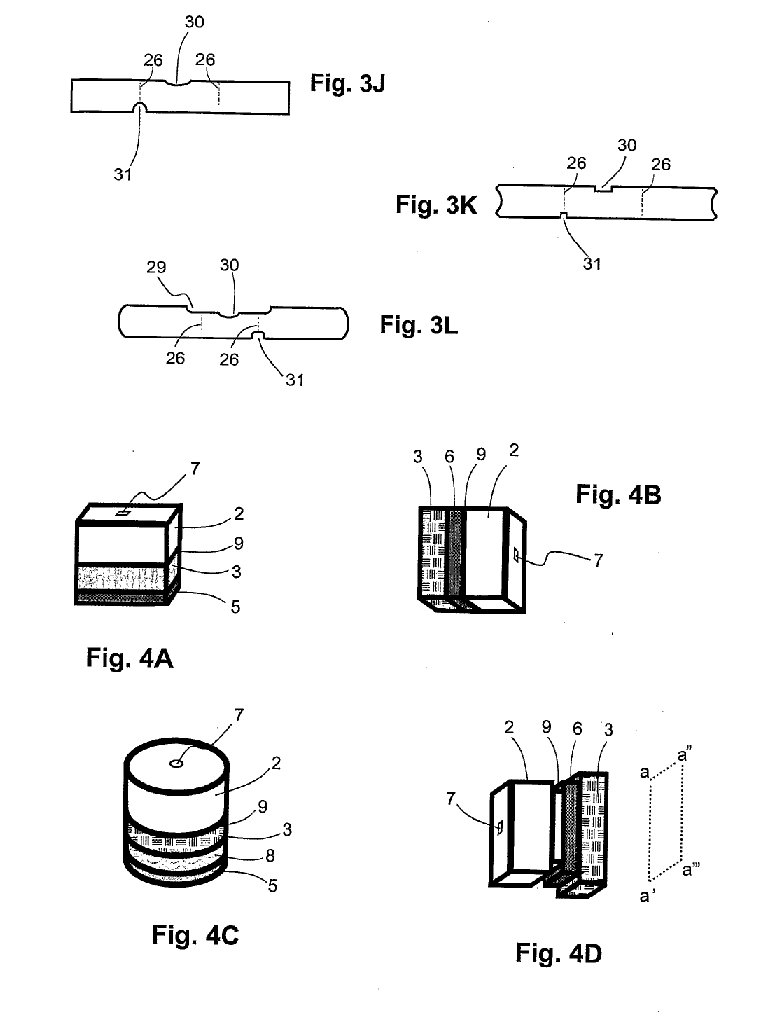

FIG. 3 shows embodiments of the sealing layer. FIGS. 3A-3I are a top view. Perforation lines 26 as well as a notch 30 allow for a mechanical penetration. FIGS. 3A to 3C show embodiments where the perforation lines are arranged in form of a cross-shape. The embodiment shown in FIG. 3C contains a pair of such perforation lines arranged in form of a cross-shape. FIG. 3D shows an embodiment where four perforation lines form the shape of a rectangle. The embodiment depicted in FIG. 3E contains three separate perforation lines of circular shape. The embodiments shown in FIGS. 3 F-I include hinges 27, which facilitate penetration. FIG. 3F shows an embodiment where perforation lines 26 form three sides of a rectangle. The fourth side of the rectangle is provided by a hinge 27. The embodiment depicted in FIG. 3G contains a single perforation line 26 of semi-circular shape and one hinge 27. Two such perforation lines are combined with a straight central perforation line and two outer hinges in the embodiment shown in FIG. 3H. FIG. 3I shows a similar embodiment where the perforation lines are arranged in H-shape. FIGS. 3J to 3L are cross sections. In addition to a notch 30 facing the sample processing chamber, the sealing layer may contain an additional notch 31 facing the absorption layer. The embodiment shown in FIG. 3 L contains a recess 29. A perforation line 26 and a notch 30 are comprised within this recess 29. It should be understood that in some embodiments the absorption layer may be manufactured in a similar manner as the depicted embodiments of a sealing layer.

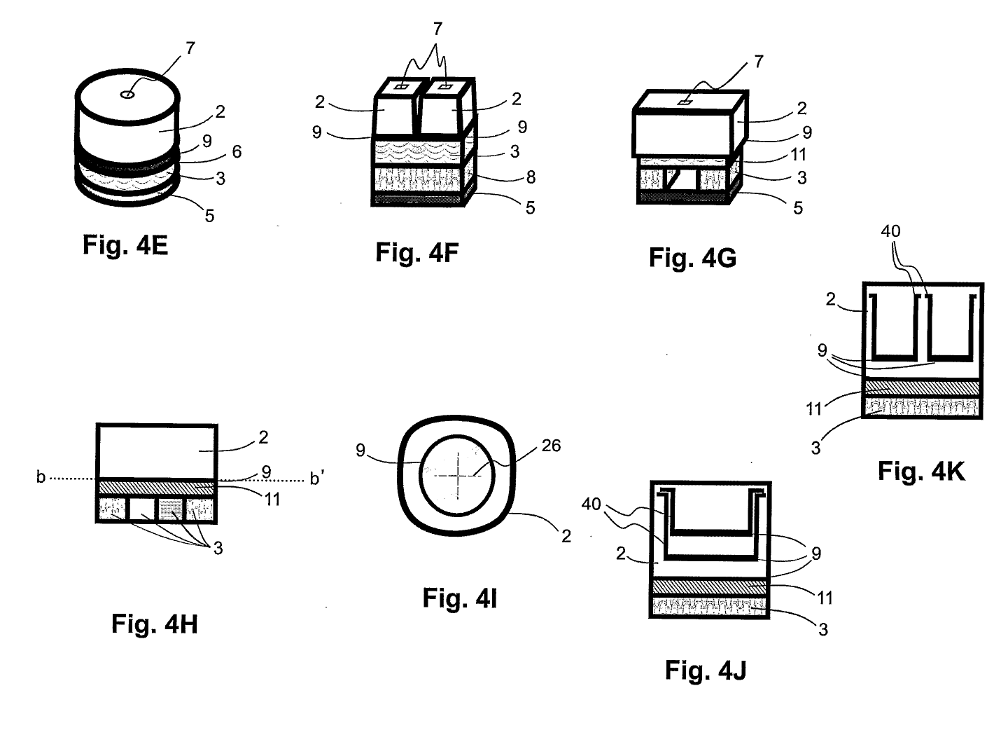

FIG. 4 shows further embodiments of a device of the invention. FIGS. 4A, 4C, 4E, 4F and 4G depict an additional supportive layer 5 contacting the absorption layer on the side opposing the sealing layer. The sealing layer 9 and the supportive layer 5 may be configured to provide separation and to isolate the absorption layer. Such isolation of the absorption layer may prevent it from getting into contact with the ambience, for instance for reasons of safety and/or to prevent contamination.

FIGS. 4B, 4D and 4E depict a separate sealing layer 6 in addition to sealing layer 9. FIGS. 4C and 4F depict a further absorption layer 8 in addition to absorption layer 3. FIG. 4D depicts an embodiment where the spatial extent of the sections of sample processing chamber 2, sealing layer 9, separate sealing layer 6, and absorption layer 3 differ in the plane as defined by a′-a″-a′″. FIG. 4F shows an embodiment where the device contains two sample processing chambers 2.

FIG. 4G depicts an embodiment comprising a guiding layer 11 in addition to a divided absorption layer 3, which consists of two separate parts. The guiding layer may serve the function of transmitting fluid from the sample processing chamber 2 to the absorption layer 3, once sealing layer 9 has been perforated. FIG. 4H is a side view of an embodiment that includes a guiding layer 11 and an absorption layer 3, which consists of four different parts that are arranged side by side. These different parts of the absorption layer may include or consist of different materials. FIG. 41 is a cross section of the device depicted in FIG. 4 H at the line b-b′. The cross section shows that the sample processing chamber 2 extends further than the sealing layer 9 in the dimension of line b-b′. Thus the sealing layer forms a part of the respective wall of the sample processing chamber 2. FIGS. 4J and 4K show two embodiments of the device that contain a guiding layer 11 and a homogenous absorption layer 3. They furthermore possess two sub-chambers 40, which are located inside the sample processing chamber 2. Both sub-chambers contain a sealing layer 9. Any of the layers depicted in the Figure, such as for example sealing layers, guiding layers or absorption layers, may be detachable.

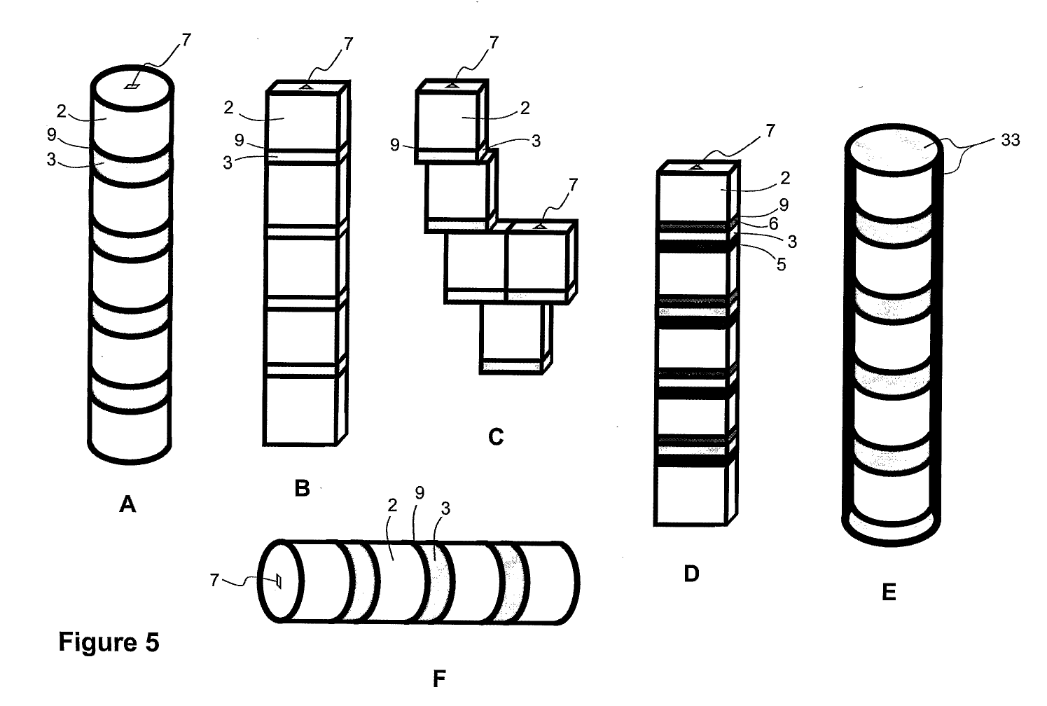

FIG. 5 shows arrangements of several devices of the invention, which are adapted to be securable to another device, such that the outlet of one device is in fluid communication with the inlet of another device. The arrangement shown in FIG. 5C includes two inlets 7 that are accessible from the ambience. FIG. 5E depicts an embodiment, where devices are comprised in a housing. FIGS. 5A to 5E depict embodiments where the devices of the invention are arranged with an orientation of the absorption layer 3 vertically below the inlet 7 of the sample processing chamber. FIG. 5F depicts an embodiment with a horizontal orientation of the absorption layer 3 and the inlet 7 of the sample processing chamber, i.e. where several devices are arranged with their inlets horizontally side by side.

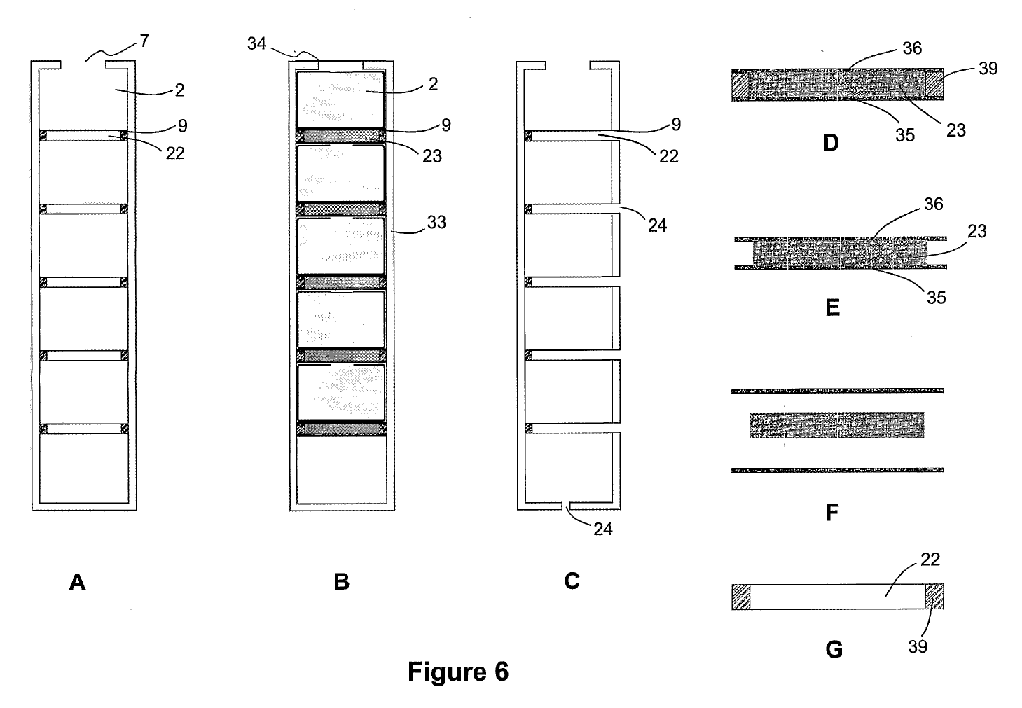

FIG. 6 shows embodiments of a device including several sample processing chambers 2, sealing layers 9 and absorption layers 23. FIG. 6A shows an embodiment where these sample processing chambers 2 and sealing layers 9 are merged into one compact unit. The absorption layers 23 may be detachable, thus leaving a space 22 for housing them, as shown in FIG. 6A. FIG. 6B shows an embodiment, where the sample processing chambers 2, the sealing layers 9 and absorption layers 23 are comprised in a housing 33. The chambers (together with the sealing layers) as well as the absorption layers may be detachable. The absorption layer may thus for instance be replaced by an absorption layer 23 of a specially selected material (FIG. 6B). The housing may possess one or more openings 24 as shown in FIG. 6C. Such openings may serve in exchanging the absorption layer. They may also be used for introducing or removing material, e.g. a fluid or recovered target matter. Optionally these openings may be sealable (not shown). The seal 34 of inlet 7 may be an integral part of the compact unit or detachable.

FIGS. 6 D-G show a close-up view of the elements surrounding absorption layer 23 for better view. In the embodiments depicted in FIGS. 6A-C the absorption layers 23 is of smaller vertical dimension (i.e. width) than the vertical dimension of chamber 2 and sealing layer 9. Therefore the respective devices contain a spacer 39, which assists in supporting chamber 2 and sealing layer 9. The devices also contain a multifunctional layer 35, which may serve as an additional sealing layer and/or as a wall of space 22 (FIG. 6G), thus allowing for an exchange of absorption layer 23.

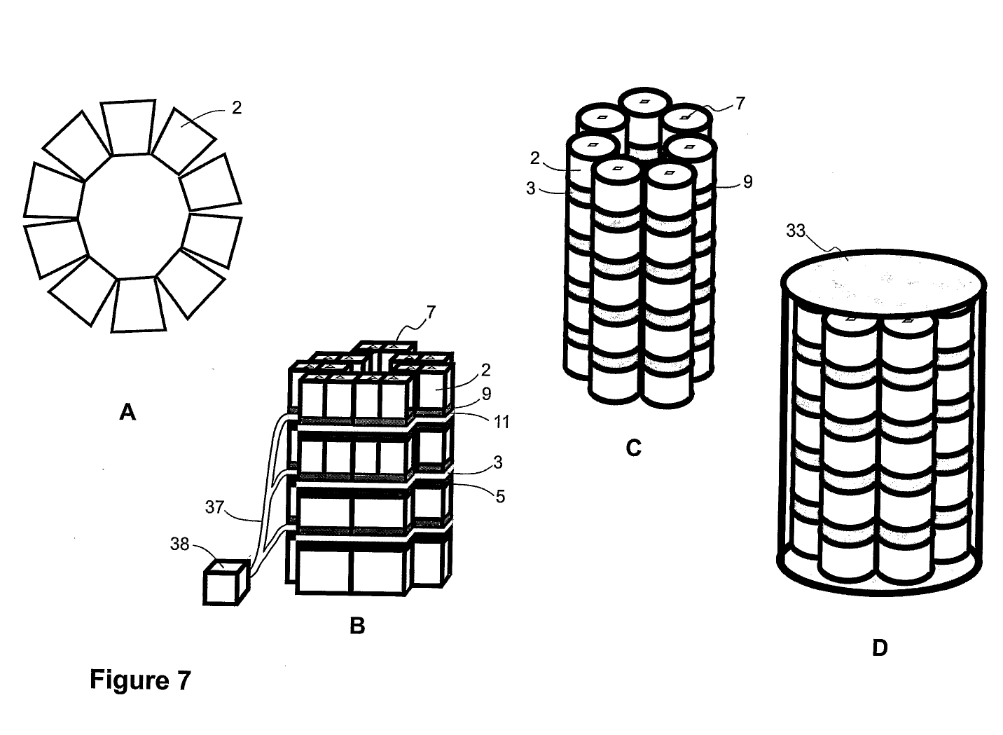

FIG. 7 shows embodiments of an arrangement within one device or of several devices of the invention. FIG. 7A depicts the arrangement of several sample processing chambers, which are adjoined side by side. Several devices or other elements of the device, such as sealing layers 9 and/or absorption layers 3 may be arranged accordingly. Thus, in some embodiments FIG. 7A represents an arrangement of several individual devices, adjoined side by side. In other embodiments FIG. 7A represents an arrangement of several sample processing chambers, which are comprised within a single device. For some of the embodiments outlined above, FIG. 7A represents a view along the axis of the inlets. Such embodiments are depicted in FIGS. 7B-D.

FIG. 7B depicts an embodiment of a device that contains multiple sample processing chambers, sealing layers and absorption layers. The absorption layers 3 stretch through the entire horizontal dimension of the device. In contrast thereto, several sample processing chambers 2, guiding layers 11, and additional supportive layers 5 are arranged in the horizontal dimension. The embodiment shown in FIG. 7B furthermore provides a tubing 37. This tubing establishes a direct fluidic connection between the absorption layer and a waste chamber 38. It is thus able to transfer fluid from the absorption layer to a waste chamber 38. FIG. 7C shows an embodiment where several devices are adjoined in two different dimensions. In the horizontal dimension, as defined by the level of the inlets 7 and any level parallel thereto, the devices are arranged in a manner reflecting the arrangement depicted in FIG. 7A. In the vertical dimension the devices are stacked. Each device includes a separate sample processing chamber 2, a sealing layer 9, and an absorption layer 3. A respective arrangement of devices may be comprised in a housing as shown in FIG. 7D.

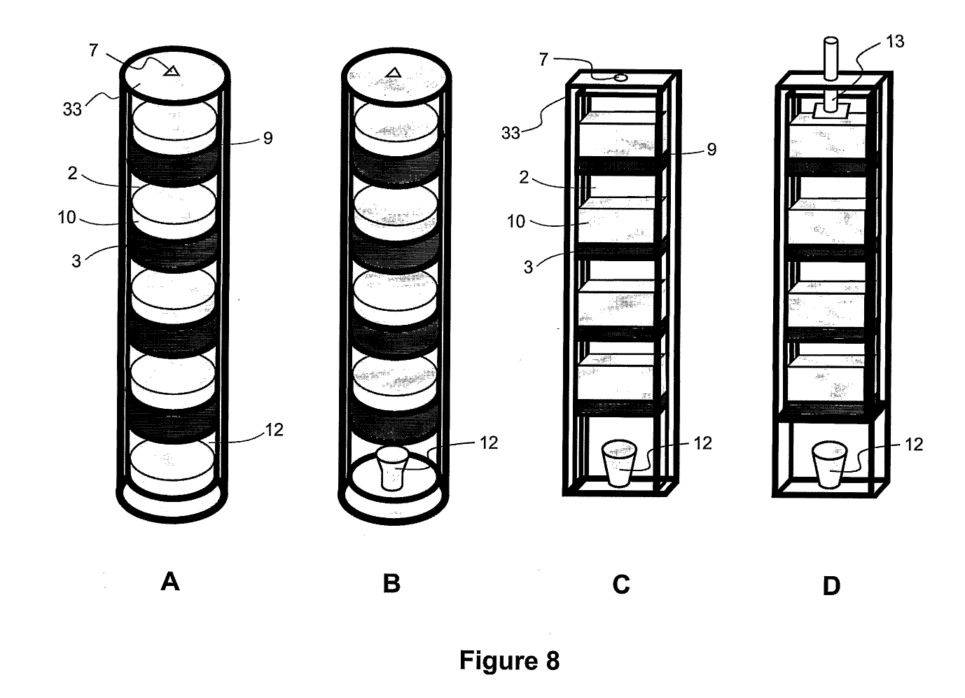

FIG. 8 depicts an apparatus comprising several devices of the invention. The devices contain a medium 10 and are comprised in a housing 33. The apparatus contains an elution chamber 12. This elution chamber may be of the same form and dimensions as the sample processing chambers as shown in FIG. 8A, or different as shown in FIG. 8B-D. FIG. 8 D further shows a probe 13 inserted into a sample processing chamber.

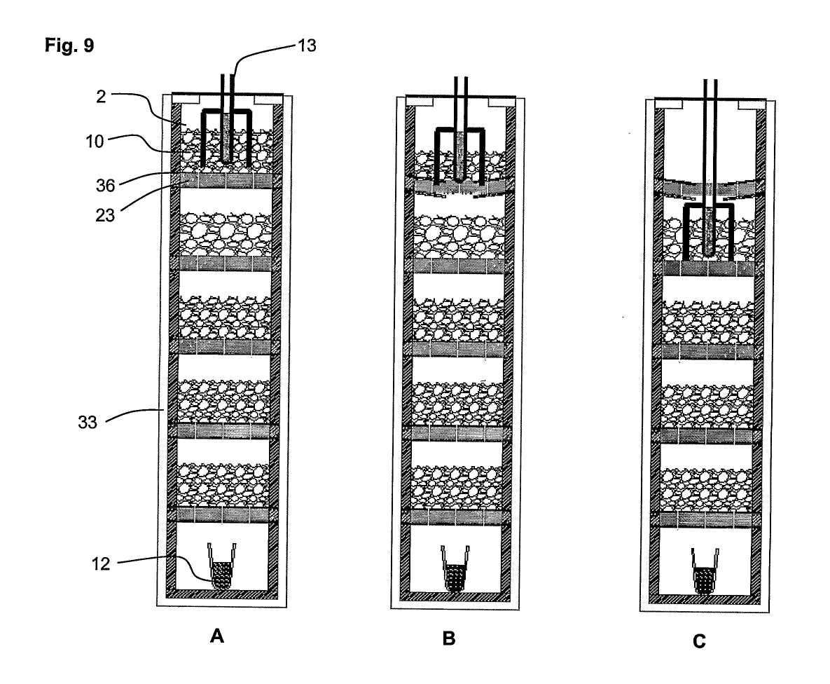

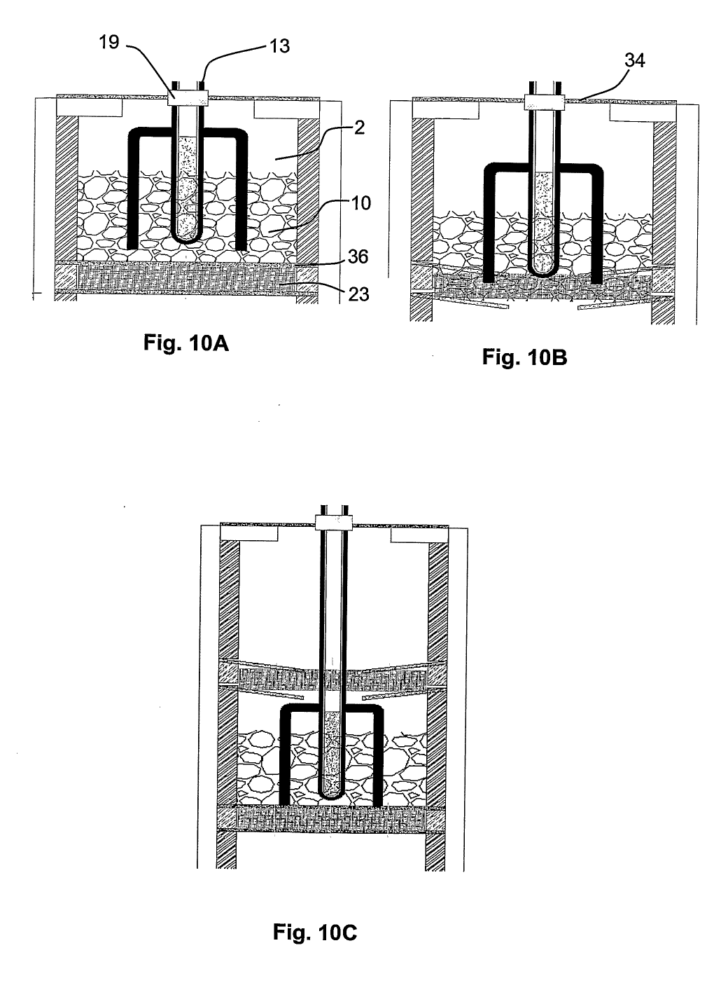

FIG. 9 illustrates the use of an apparatus as shown in FIG. 8. Initially the probe 13 is used for recovering a sample in the first sample processing chamber (FIG. 9A). Subsequently sealing layer 36 is penetrated by means of the probe (FIG. 9B). At this time the sample is bound to the probe. The absorption layer 23 (comprised in housing 33) now absorbs medium 10. Thereafter probe 13 penetrates the absorption layer (FIG. 9C). It is thus used for transferring the sample to the next sample processing chamber.

FIG. 10 depicts a close-up view of the steps of FIG. 9. It furthermore shows an interface 19, which seals the highest sample processing chamber 2 while the probe is inserted therein. The inlet of the sample processing chamber may be externally accessible and covered by a seal 34. As this seal includes interface 19, it seals the device.

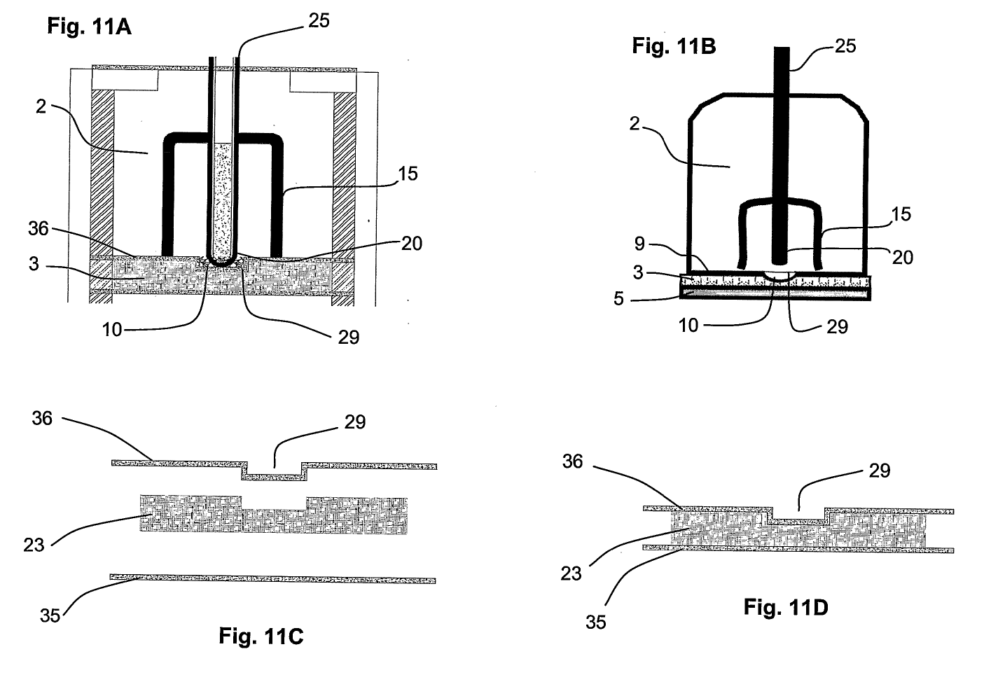

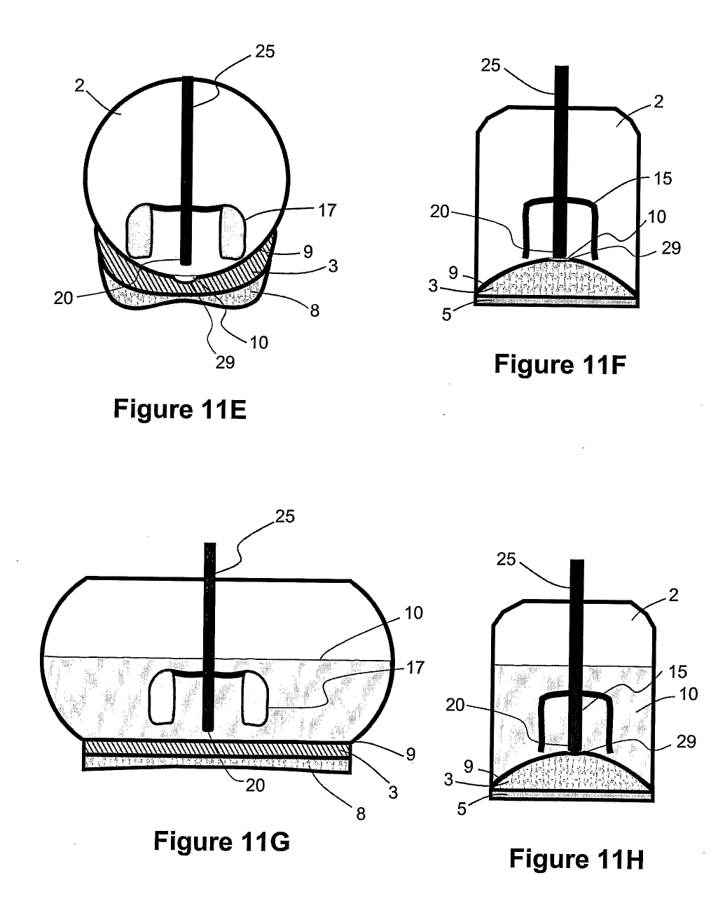

FIG. 11 depicts embodiments of a device of the invention that contain fluid 10. The embodiments shown in FIGS. 11A, 11B, 11E, 11F and 11H include a sample processing chamber 2 that can comprise a very small amount of a fluid 10 in a recess 29 of a sealing layer. The view of the processing chamber 2 and the probe in these figures shows that sample processing chamber 2 provides sufficient space for accommodation of more fluid if required. The same steps as shown in FIG. 10 can be performed. FIG. 11A depicts a recess 29 of a sealing layer 36 accommodating fluid 10. In this embodiment contacting the sealing layer are probe arms 15 and not its head 20. This may allows for substantial shielding of the head 20 and any bound matter (not shown). FIG. 11B depicts an embodiment where the sample processing chamber 2 contains a sealing layer 9 with a recess 29, filled with fluid 10. The head 20 of the probe does not immerse into fluid 10 when the arms 15 contact the sealing layer 9 since arms 15 extend further than the head of the probe. Thus the arms 15 will penetrate sealing layer 9 first. Subsequently head 20 will immerse into recess 29 and thereafter penetrate the sealing layer 9. FIGS. 11C and 11D show an exploded view of sealing layer 36 with recess 29, absorption layer 23 and a supportive part 35 of the housing of the device shown in FIG. 11A, disassembled and assembled respectively.

FIG. 11E shows an embodiment with a round sample processing chamber 2, a probe with a hollow primary core rod (sheath) 25, and blades 17. Sealing layer 9 possesses a recess 29 filled with fluid 10. Below the sealing layer are an absorption layer 3 and a second absorption layer 8. Although the head 20 of the probe extends further than blades 17, the blades 17 of the probe will penetrate sealing layer 9 first. The device depicted in FIG. 11F has a convex sealing layer 9 on top of a flat additional supportive layer 5. The strength of this sealing layer increases from the center accommodating the recess 29 toward the external areas, at which the figure labels are located. The sealing layer is thinnest at recess 29. Although the arms 15 of the probe extend further than the head 20, the head 20 will penetrate sealing layer 9 first.

FIG. 11G depicts an embodiment of the device, where the head 20 and the blades 17 of the probe are submerged in fluid 10. Below the sealing layer 9 (without a recess) are an absorption layer 3 and a second absorption layer 8. The design of the probe resembles the design shown in FIG. 11E. Due to the different design of the sample processing chamber however, the head 20 will penetrate sealing layer 9 first. FIG. 11H depicts the embodiment of the device shown in FIG. 11F. Contrary to FIG. 11F, where only recess 29 is filled with fluid 10, FIG. 11H shows a state where the head 20 and the blades 17 of the probe are submerged in fluid 10.

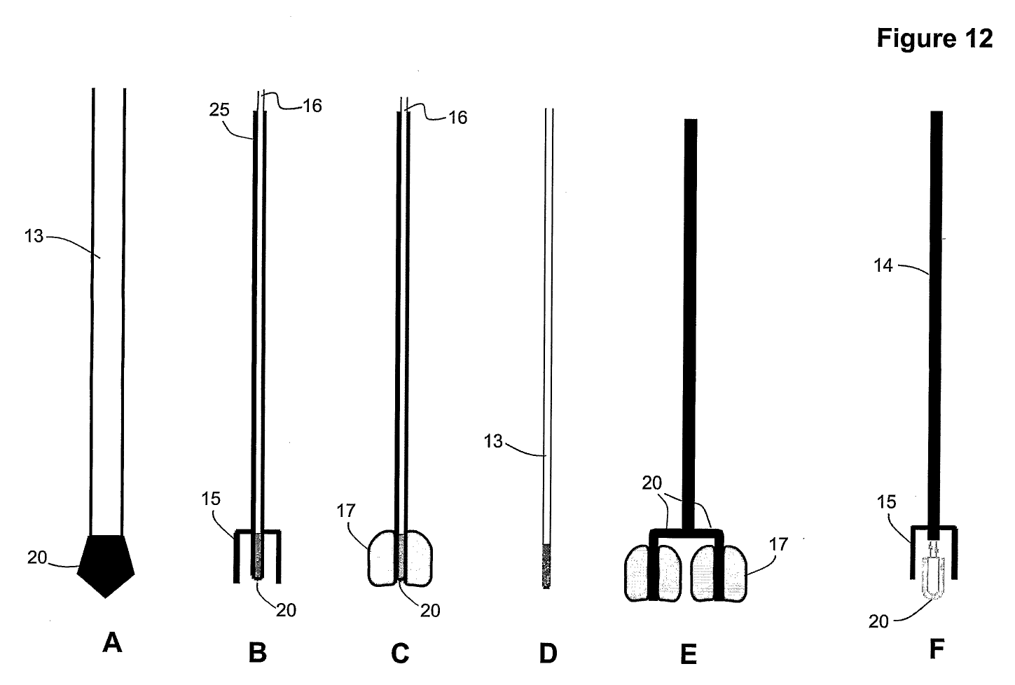

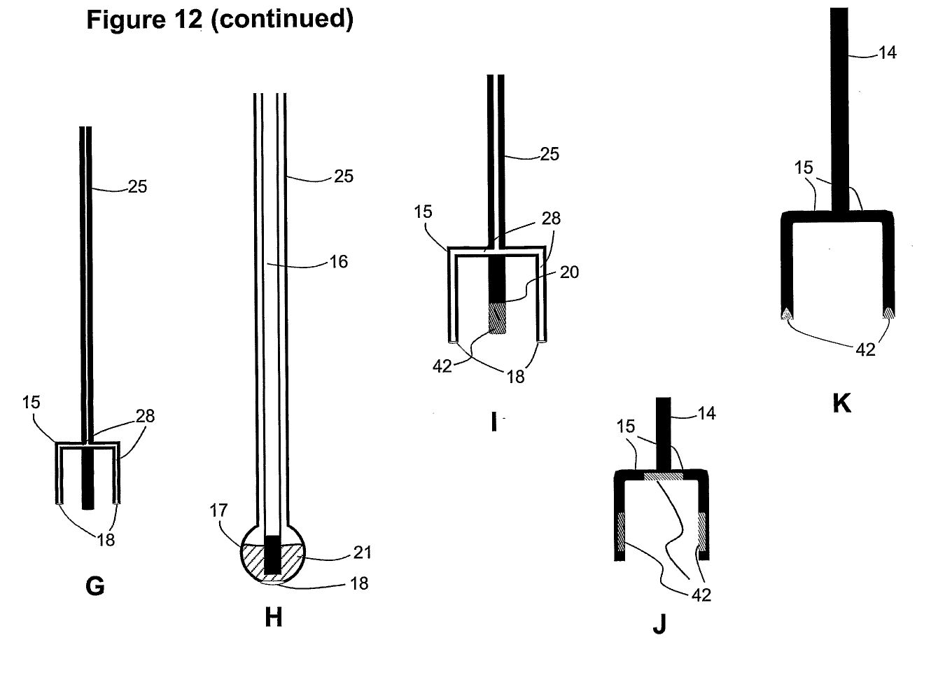

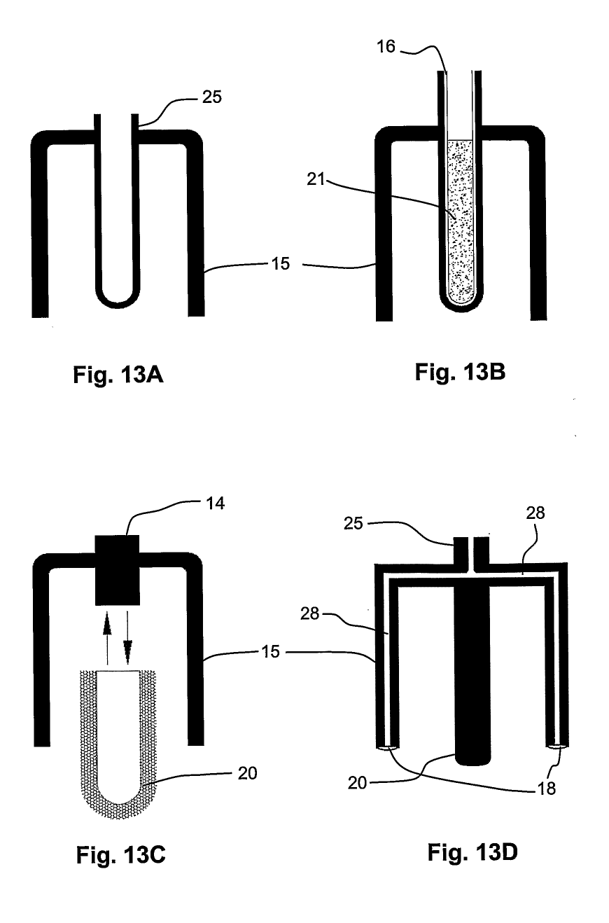

FIG. 12 depicts different embodiments of probe 13. A: The probe is a compact unit ending in a head 20. B: The probe contains a hollow primary core rod 25, which contains a removable core rod 16, which is hollow core in this embodiment. It further includes legs 15. C: Probe 13 contains a core rod 16 and includes blades. 17. D: Probe 13 is a hollow housing that may contain fluid. E: The head 20 of the probe consists of several connected portions, each of which in turn includes blades 17. F: Probe 13 includes a primary core rod 14 and a removable head 20. G: The hollow primary core rod 25 of the probe as well as arms 15 include a channel 28 ending in sealed openings 18. H: The probe 13 contains a hollow primary core rod (outer sheath) 25, a core 16 and includes hollow blades 17 that may comprise a fluid. The fluid may be released via a sealed opening 18. I: The hollow primary core rod 25 and the arms 15 of the probe include a channel 28 ending in sealed openings 18. The head 20 of the probe is equipped with a binding region 42. J: The probe contains primary core rod 14 and legs 15. Certain surface area of the legs 15 are rendered with an affinity for selected target matter. They thus form different binding regions 42. K: In this embodiment the ends of legs 15 of the probe are equipped with different binding regions 42.

FIG. 13 depicts a close-up view of the embodiments shown in FIGS. 12 B, F and G. FIG. 13B shows that core 16 of the probe may contain fluid 21. FIG. 13C shows an embodiment where head 20 is removable. A respective head may for instance contain a fluid. FIG. 13D shows an embodiment where the central rod and the arms 15 include a channel 28. The channel may contain a fluid, which may be released through sealed openings 18.

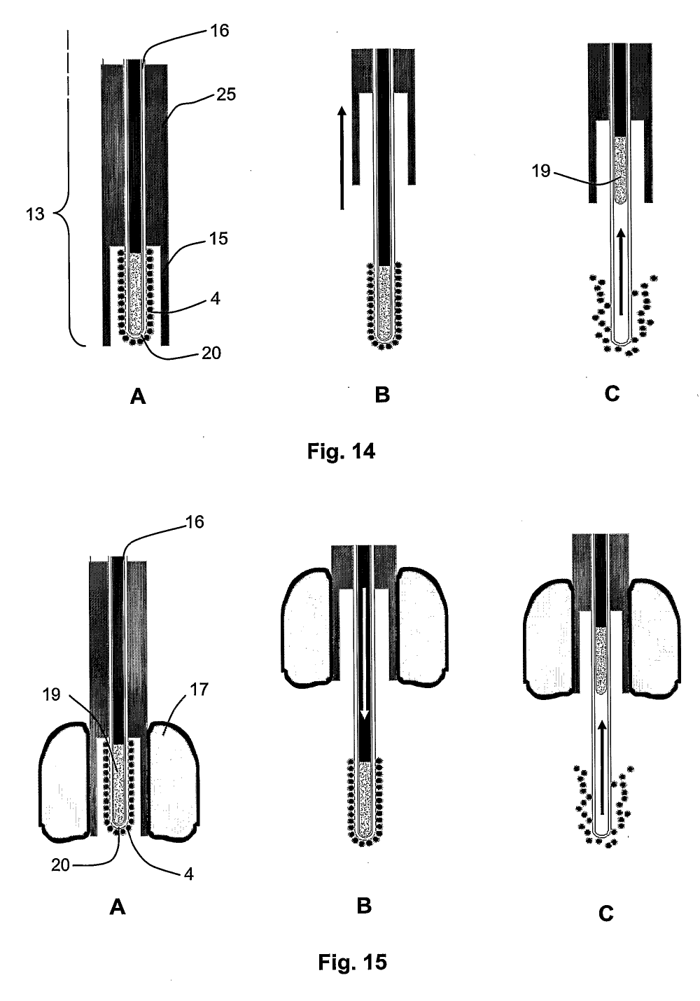

FIG. 14 depicts a release of magnetic beads 4 from a probe 13 with retractable arms 15 and a rod 14 of large diameter. FIG. 14A shows a conformation where the arms 15 are in a protective position, shielding the magnetic beads 4, which are bound to head 20 of the probe 13. In FIG. 14B the arms 15 with the rod are in upward motion, thus lifting the shield. In this position, magnetic beads 4 are easily bound to the probe. FIG. 14C shows an upward motion of the core rod 16 of the probe. In this embodiment the core rod 16 comprises a magnetic core 19, so that its removal leads to the release of magnetic beads 4 from the probe 13.

FIG. 15 depicts a release of magnetic beads 4 from a retractable probe 13. FIG. 15A shows a conformation where the blades 17 are in a protective position, shielding the magnetic beads 4, which are bound to the probe. This position may also serve as the collecting position, where magnetic beads 4 are being attracted to the head 20 of the probe. FIG. 15B shows a downward motion of the core rod 16, thus lifting the shield. In this position, magnetic beads 4 are easily accessible and can thus be easily released from the probe. FIG. 15C shows an upward motion of the core rod 16 of the probe resulting in a release position. In this embodiment the core rod 16 comprises a magnetic core 19, so that its removal leads to the release of magnetic beads 4 from the probe.

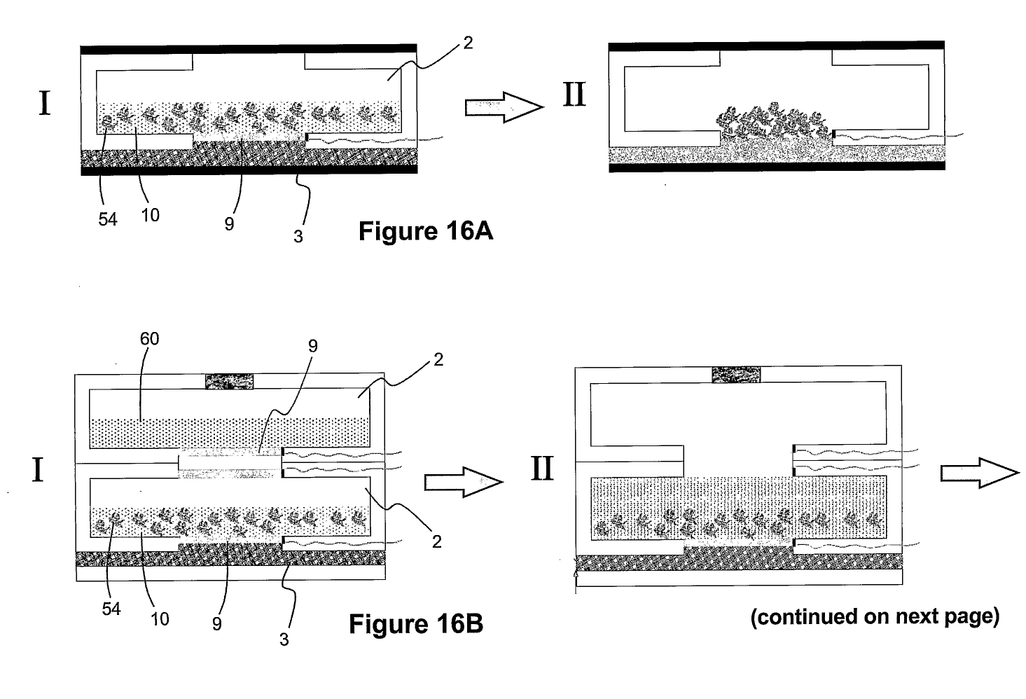

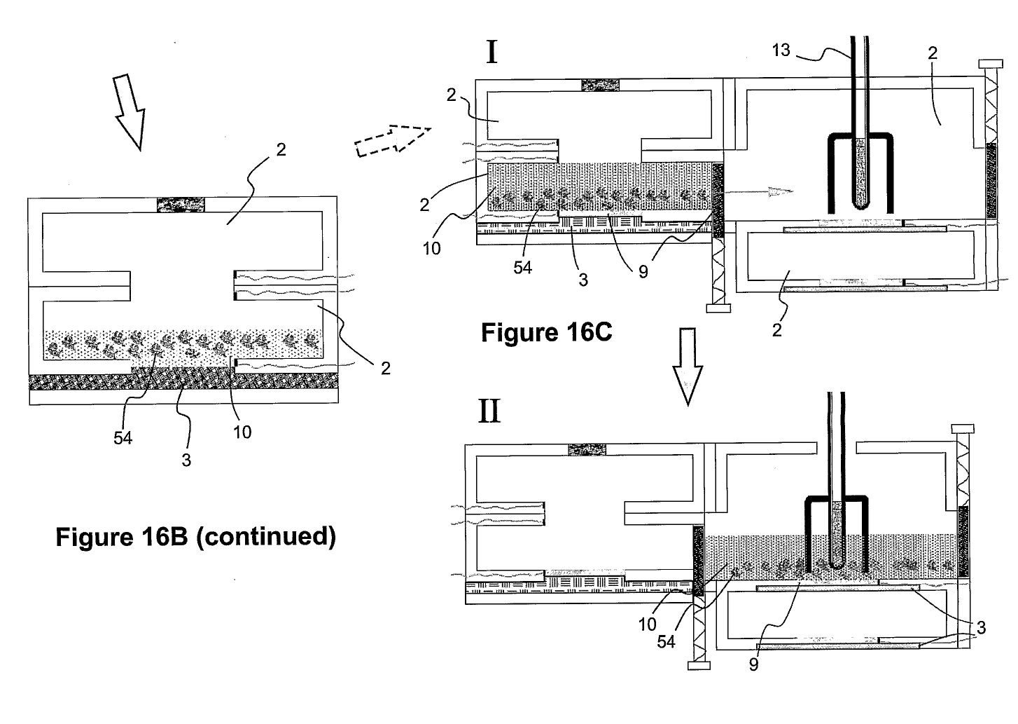

FIG. 16 shows exemplary embodiments of the method of the invention. FIG. 16A shows on the left a fluid 10 comprised in a sample processing chamber 2. Solid target matter 54 forms a suspension in the fluid. Sealing layer 9, which consists of a smart material, is then caused to be penetrated (II, right half), so that absorption layer 3 accommodates fluid 10. Target matter is not able to enter absorption layer 3 and is therefore separated from both the fluid and the absorption layer.

FIG. 16B shows a device that includes two sample processing chambers 2. The lower chamber contains a suspension of solid target matter 54 in fluid 10. The upper chamber contains a further fluid 60, which may contain further matter such as reactants, reagents etc. Sealing layer 9 of the upper chamber is then caused to be penetrated (II, on the right), so that fluid 60, which was contained in the upper chamber, is being accommodated in the lower chamber. As a result a process such as a chemical reaction may take place. Thereafter sealing layer 9 of the lower chamber is caused to be penetrated (III, bottom of next page), so that absorption layer 3 which has selective absorptivity for fluid 60 absorbs fluid 60. Target matter 54 and fluid 10 are not able to enter absorption layer 3 and are therefore separated from both fluid 60 and the absorption layer.

FIG. 16C shows a device that includes four sample processing chambers 2. In the state shown on the top (I) the lower chamber on the left contains a suspension of solid target matter 54 in fluid 10. As indicated by an arrow, this state may be the first step of a sequence of steps or alternatively the continuation of a sequence of steps depicted on the previous page. In the latter case the depicted state is a result of the penetration of a sealing layer between the two sample processing chambers on the left side of the device as illustrated in steps I, II and III of FIG. 16B.

Between the lower chamber on the left and the chamber on the right side of this chamber (into which a probe 13 is inserted) there is a sealing layer 9. This sealing layer can form an opening in a reversible manner. Thus the sealing layer may then be opened. Pressure may be applied or the device may be rotated clockwise (around the axis defined by the respective sealing layer), so that fluid 10 together with target matter 54 is moved into the depicted upper sample processing chamber 2 on the right, as shown in step II (lower part of FIG. 16C). Resealing of sealing layer 9, located between this chamber and the chamber where the fluid was located in step I (as described above), may be performed. Such resealing prevents fluid 10 and target matter 54 from flowing back. Probe 13 may now, or in a subsequent step, be used to attract target matter 54 (not shown).

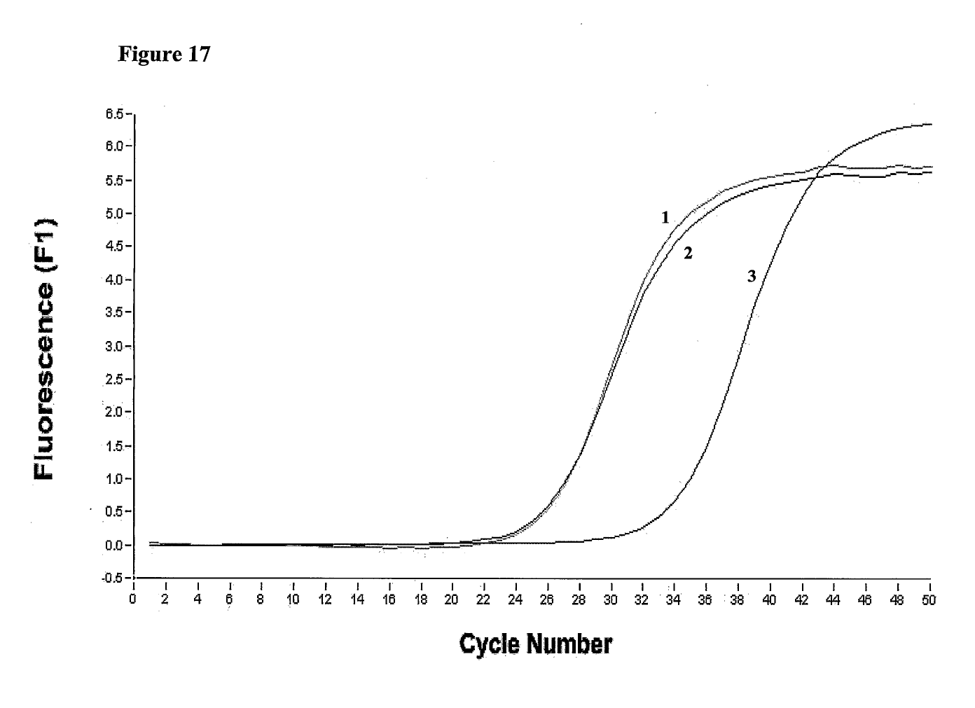

FIG. 17 shows the amplification analysis in a PCR machine by real time detection. Dengue RNA was extracted in the device of the invention using magnetic beads. 1: first extraction using the device of the invention; 2: second extraction using the device of the invention; 3: autoclaved deionised water.

FIG. 17 shows that fluorescence signals generated from the isolation product obtained with the device of the present invention appear substantially faster than the signal of the negative control (cf. also the Ct values in Example 4). This indicates successful amplification of isolated RNA.

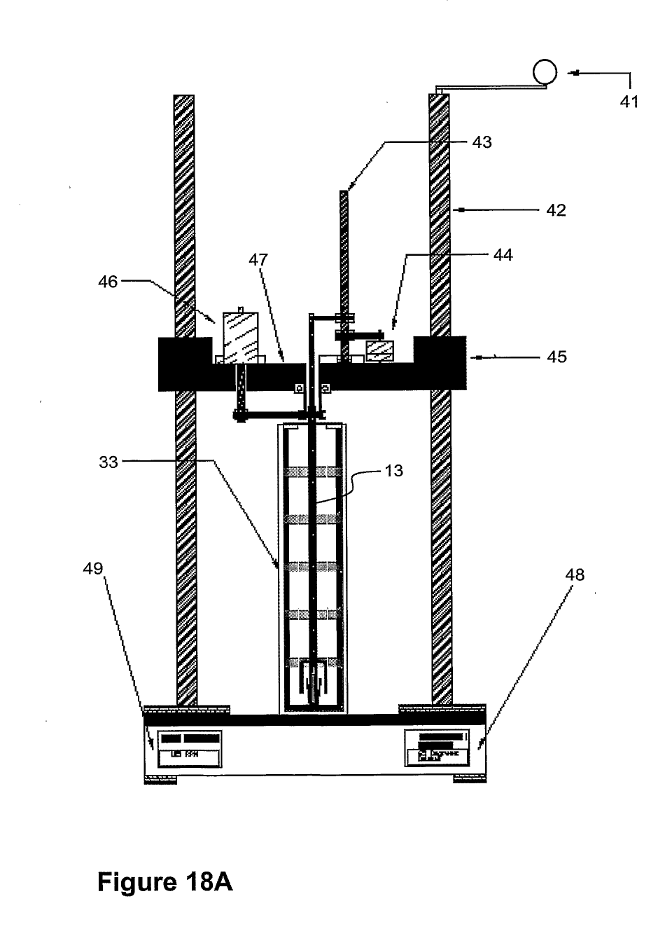

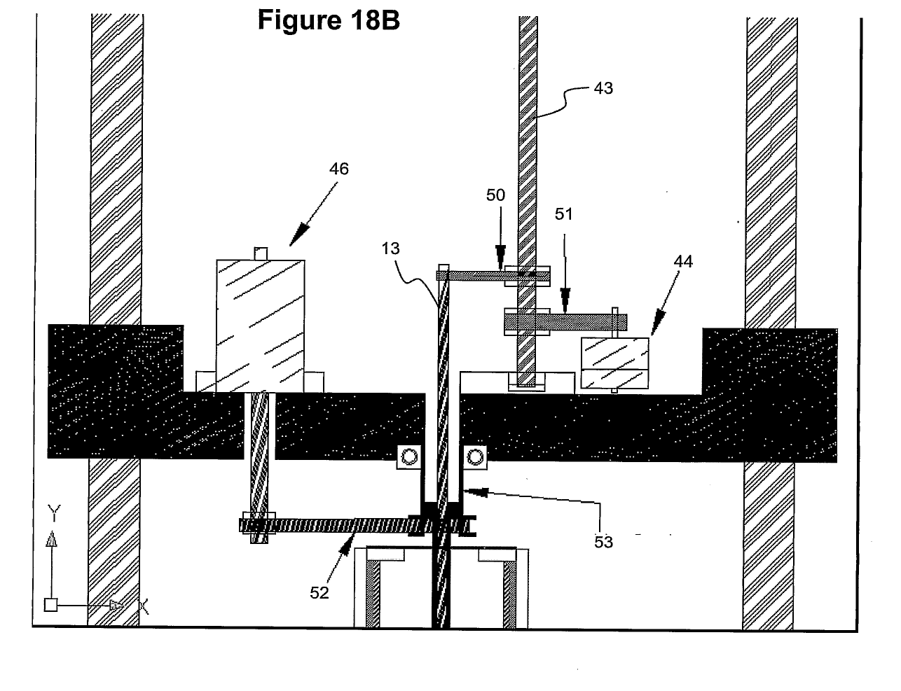

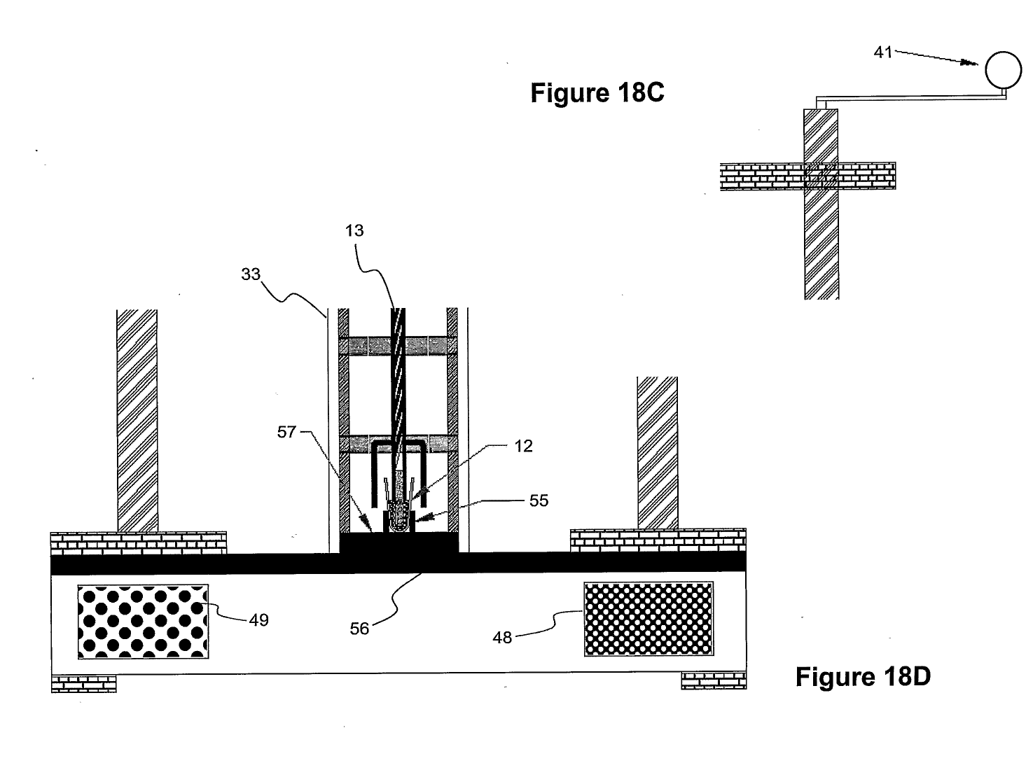

FIG. 18A depicts an example of a configuration of an apparatus of the present invention. The device resembles the device depicted in FIG. 9 and includes several sample processing chambers and a housing 33, is shown in the center of the figure. An agitation motor 46 and an actuator motor 44 are together with a miniaturised lead screw 43 able to control the probe 13 inserted into the apparatus. The agitation motor 46 and the actuator motor 44 are mounted onto a base plate 47. A lead screw 42 together with a ball screw 45 serves as a linear motion guide, which controls vertical movements. By means of a wheel handle 41 the leadscrew 45 can be turned clockwise or counterclockwise, thus moving the ballscrew up or down. The ball screw is connected to the base plate 47, to which the motors are attached (cf. above).

FIG. 18B is an enlargement of the central part of the configuration depicted in FIG. 18A. As can be seen, the probe is secured by a holder 53. A belt 52 connects to the probe holder to the agitation motor. A further belt 50 connects the probe of the apparatus to the miniaturized lead screw 43. The miniaturized lead screw is connected to the actuation motor 44 for retracting and lowering the probe 13 by a belt 51.

FIG. 18C depicts an enlargement of the part of the configuration depicted in FIG. 18A that contains the wheel handle 41 for better view.

FIG. 18D depicts an enlargement of the lower part of the configuration depicted in FIG. 18A. The bottom of the apparatus is formed by an aluminium base plate 57, which is an integral part of the housing 33 of the apparatus. An aluminium holder secures elution chamber 12, into which the probe 13 can immerse. Located below the aluminium base plate 57 is a hotplate 56. Temperature controller 48 and speed controller 49 are located on the bottom of the configuration for convenience.

### EXAMPLES

### Example 1

### Fabrication and Assembly of a Device with Five Chambers

The present example illustrates the manufacture of an exemplary device of the present invention. The exemplary device contains five chambers, of which four are designed for usage as sample processing chambers and one to house an elution chamber.

### a) Fabrication of the Sample Processing Chambers

A 32.0 mm×32.0 mm square transparent acrylic block (Perspex) with the thickness of 20.0 mm was selected to fabricate the processing chamber. The shape of the sample processing chamber was formed by CO₂ laser drilling, such that a hole of 15.0 mm diameter was generated in the center of the square block. This hole formed part of the interior of the sample processing chamber (to be completed by the sealing layer) and its diameter was determined to have a capacity of more than 2.0 ml volume. The inlet of the sample processing chamber was formed on the second large side of the block, opposing the side where the hole had been formed. This inlet had the form of a second hole of a diameter of 2.0 mm. This inlet thus allows the injection of fluid and other matter required for processing. Alignment holes of 3.0 mm diameter were also laser drilled on the four corners of block (i.e. not contacting the chamber), when seen from the top of one of the two large sides, for example from the inlet. These alignment holes later facilitated accurate alignment during assembly.

### b) Fabrication of the Penetrable Sealing Layer

A penetrable plastic sealing layer was made of acrylic material obtained from 3M with a thickness of 0.025 mm to 0.05 mm. The quality of this material is similar to those materials used in projector transparencies. The lateral dimension of the penetrable plastic sealing corresponded to the respective dimension of the preformed sample processing chamber (15.0 mm diameter, see above), so that the sealing layer would complete the chamber. A perforation line with a cross-shape as shown in FIG. 3B was made on the sealing layer. The center of the cross-shape was positioned to align with the center of the hole that had been formed as described above to preform the interior of the processing chamber. The perforation area was coated with a thin layer of silicon sealant for temporary sealing and protection.

### c) Fabrication of a Supportive Layer (Forming a Housing for the Absorption Layer)

A 32.0 mm×32.0 mm square transparent acrylic block (Perspex) with a thickness of 4.0 mm was selected for the supportive layer. A recess was fabricated in the supportive layer, so that the recess is able to accommodate the absorption layer. Hence, the supportive layer forms a housing for the absorption layer. The recess was again created by means of CO₂ laser drilling. An internal square shape was excised out of the block thus forming the recess to house the absorbing material. Thus one side of the housing was open and was later (see below) completed to form a hollow space by placing the sealing layer on top of it. A 3.0 mm diameter alignment hole was also CO₂ laser drilled on each of the four corners of block, when seen from the top of one of the two large sides, with the same alignment coordinates as the processing chamber.

### d) Fabrication of an Absorption Layer

The penetrable absorption layer was made of nitrocellulose material that was obtained from Whatman. Two sublayers of the nitrocellulose material were prepared, the first one 2.0 mm thick, and the second one 0.2 mm thick. The lateral dimension of both sublayers was cut to the same dimension as the inner dimension of the housing described in (c), in order to allow the two sublayers to fit exactly into the housing. A hole with a diameter corresponding to the sealing layer (and thus the sample processing chamber) was formed in the first subabsorption layer of 2.0 mm thickness. A perforation line with a cross-shape as shown in FIG. 3B was generated in the second subabsorption layer of 0.2 mm thickness. The center of the cross-shaped perforation line was positioned to align with the center of the cross shape of the penetrable sealing layer.

### e) Fabrication of a Chamber to House the Elution Chamber

A chamber resembling the sample processing chamber was manufactured as described above. A 32.0 mm×32.0 mm square aluminium block with a thickness of 8 mm was prepared. This block was used to seal the opening of 15.0 mm diameter (see above) instead of the sealing layer used above. Aluminium material was selected to afford heating and cooling at a faster rate without mechanical deformation. The chamber contains an extruded funnel shape chamber wherein the elution chamber can be placed (cf. e.g. FIG. 18C). A conventional microtube, such as an Eppendorf microtube, can thus be conveniently used as an elution chamber and reversibly be placed into the chamber of dimensions matching the sample processing chambers.

### f) Assembly of the Device of the Invention

A 5.0 mm thick stainless steel fixture with dimension of 100.0 mm×50.0 mm was used as a base for the assembly. A long stainless steel rod with the diameter of 2.0 mm and a length of 200.0 mm was mounted on the four corners of the fixture. The rods served as the skeletal frame of the device. Hence, the mounting location of the rods is constrained to exactly align with the four corner holes of the sample processing chambers, penetrable sealing layers, and supportive layers (the absorption layer is smaller with respect to its width and does not contain alignment holes).

The assembly starts with mounting onto the stainless steel fixture the chamber that serves to house the elution chamber. The top opening of this chamber was then covered by a penetrable sealing layer using a silicon glue.

A housing for the absorption layer (see above) was mounted on top of the chamber with the sealing layer. The two sublayers of the absorption layer were inserted separately. The 2.0 mm thick subabsorbtion layer was inserted first. Thereafter the subabsorbtion layer of 0.2 mm was placed on top of the subabsorbtion layer of 2.0 mm.

The penetrable sealing layer completes both the housing of the absorption layer, i.e. turns the supportive layer into a housing, and completes the sample processing chamber. It can thus principally be attached to either element first. For the assembly of the device, a sealing layer was used to complete the sample processing chamber in a first step. For this purpose two sealing layers were used for each chamber. One sealing layer was applied to the remaining opening of the sample processing chamber as described above. A second sealing layer was applied to the inlet of the sample processing chamber. The sealing layers were fused to the chamber using silicon glue. With the penetrable sealing layer already fused thereto, the processing chamber was mounted on top of the absorption layer. Hence, the housing of the absorption layer was completed in this step. In this configuration, the sample processing was on top of the absorption layer, which was located on top of the supportive layer (forming a housing for the absorption layer), and the supportive layer was on top of the chamber that serves to house the elution chamber, as for instance depicted in FIG. 4C.

The cycle of layering a supportive layer, an absorption layer, and a sample processing chamber with a sealing layer at its lower end was repeated three times. The device was ready to be used for four processing steps and one elution step.

### Example 2

### Probe Fabrication

The present example illustrates the manufacture of an exemplary probe in the manufacture of an exemplary apparatus of the present invention.

### a) Fabrication of the Hollow Primary Core Rod (Outer Sheath) of the Probe

The hollow primary core rod (outer sheath) of the probe was formed as a cylindrical tube, and made up of high density polyethylene material. The length of the tube was about 150 mm with an outer diameter of 4.0 mm and an internal diameter of 2.5 mm. The head of the probe, which can accommodate an internal magnetic core, together with the arms was fabricated in a separate step as an end-part by injection moulding. The hollow primary core rod and the end-part were joined by glue.

### b) Fabrication of the Core Rod with a Magnetic Tip

A cylindrical neodymium magnet with the diameter of 2.0 mm and a length of 5.0 mm was mounted on the tip of a 2.0 mm stainless steel rod using glue. The rod was approximately 200.0 mm long.

### Example 3

### Assembly of an Apparatus of the Invention with an Agitation Motor and a Vertical Guide (Linear Motion Using Lead Screw)

The present example illustrates the combination of an exemplary agitation motor and a device of the present invention (as described above) in the manufacture of an exemplary apparatus of the present invention. In this embodiment a motor and a vertical guide are part of the apparatus of the invention.

The probe (cf. Example 2) was inserted into the device that had been manufactured as described in Example 1. A probe holder was selected to be able to match the outer diameter of the probe. The probe holder was a hollow cylindrical device with bearings for rotation. The probe holder was used for attaching and removing the hollow primary core rod of the probe in a manner as depicted in FIG. 18B. The hollow primary core rod of the probe was attached by friction fit and was removed by pulling using minimal force. The probe holder was fixed to a stainless metal plate (see FIG. 18B). This plate was used as a base plate to mount a DC motor for agitation. The DC motor was connected to the holder of the probe by means of a belt (FIG. 18C).

The base plate with the motor and the probe holder was fixed to a vertical guide. The vertical guide was made up of lead screw (cf. FIG. 18A) with supporting frameworks of acrylic and aluminium rods. To allow for control of the speed of the motor, it was connected to an analogue tuner, the speed ranging from 10 rpm to 300 rpm. The level of the probe inside the device when processing was regulated by the vertical guide by means of a horizontal shaft. This shaft could manually be turned to achieve the require level for the intended process.

### Example 4

### Use of the Apparatus for RNA Extraction and Amplification

The present example illustrates an exemplary application of the method of the present invention. An apparatus of the invention, manufactured and assembled as described above, and including five sample processing chambers and one chamber to house an elution chamber, was used to extract Dengue virus RNA. A conventional microtube, pre-loaded with 50 μl of sterile deionised water, had been placed into the chamber to house the elution chamber (cf. Example 1e).

### a) Viral RNA Extraction

The acrylic and plastic sealing materials used in this experiment were treated overnight with 0.1 M HCl and later treated with RNase Zap buffer (Ambion Diagnostics, Austin, Tex., USA) was used to prevent contamination. The extraction of Dengue RNA was performed in three independent identical experiments.

The first of the five sample processing chambers was preloaded with 250 μl of lysis buffer and 750 ml of binding buffer. A silica coated magnetic beads with the diameter of 5 um of was introduced into the mixture. A volume of 1.0 ml of 50% ethanol with water was introduced to the sample processing chambers, which were located below the first sample processing chamber, for washing steps. For this experiment, four processing chambers for four washing steps were prepared. A volume of 50 μl of deionised water had already been introduced into an elution chamber that was located in a fifth chamber.

A 50 μl whole blood sample was prepared, and was spiked with 15,000 pfu of inactivated Dengue virus. The blood sample was injected to the first processing chamber for 15 minutes for effective lysis of the cells and binding of the nucleic acids to the silica coated magnetic beads. The plastic probe (as described in the fabrication examples), which was already situated inside the processing chamber, without the inner magnetic core on the tip was used to agitate the mixture. After the processing time was completed, the core of the probe with a magnetic end was lowered into a collection position as illustrated in FIG. 15A for 3 minutes to collect the silica magnetic beads.

The probe with the magnetic beads bound to it penetrated the sealing layer, thus establishing fluid contact of the sample processing chamber with the absorption layer. The absorption layer absorbed the supernatant released from the sample processing chamber. Penetration of the sealing layer and absorption of the supernatant occurred almost simultaneously. The penetration was controlled by the vertical guide (as describe in fabrication example 3). The probe was securely held in the required level in the next sample processing chamber. In this sample processing chamber a washing step was performed. The inner magnetic core of the probe was retracted up by 50 mm, thereby allowing the release of magnetic beads in the washing buffer for 3 minutes, before the inner probe was lowered again into a collective position (cf. FIG. 15). The penetration cycle, the re-suspension and re-collection cycle, and the washing cycle was repeated three times to purify the RNA or nucleic acids.

In the lowest chamber, which served as a housing for the elution chamber, the probe, which had the magnetic beads bound to it, entered the elution chamber, which had been pre-loaded with 50 μl of deionised water. Heating was conducted by means of the hotplate located below the elution chamber (cf. FIG. 18D). The temperature of the hotplate was set to 650° Celsius, to elute nucleic acids from the surface of silica coated magnetic beads. The eluent was then collected for characterization and evaluation.

### b) PCR Test

The eluent containing the RNA was characterized using Roche's Light Cycler, which is a Real-Time PCR Machine. A 10.0 μl volume of PCR mix was prepared with the set up reaction comprising 1× Taq buffer (Invitrogen), 0.2 mM dNTP, 4 mM MgCl₂, 0.5 mg/ml BSA, 0.2× Syber Green I, 1.25 U Platinum Taq polymerase (Invitrogen), and 1.94 μl of eluent (isolated by the method of the present invention). Autoclaved deionised water served as a negative control.

Real time detection provided an amplification plot depicting the fluorescence signal versus reaction time expressed as cycle numbers (see FIG. 16). An increase in fluorescence above the baseline indicates the detection of accumulating amplification product. Where a fixed fluorescence threshold is set above the baseline, the fluorescence signal thus passes this threshold at a certain time point. As time is expressed in terms of cycle numbers, a so called cycle threshold number (or value) or Ct value is obtained. The smaller this number, the further to the left is a respective fluorescence curve located in the amplification plot and the faster does amplification occur. The higher this number, the slower an amplification occurs and the less it becomes distinguishable from non-specific background reactions. An illustration of obtained fluorescence signals using the method of the present invention and is depicted in FIG. 17. The following Ct numbers were obtained:

| Platforms | CT (Cycle Threshold) |
|---|---|
| Present Invention Run 1 | 26.17 |
| Present Invention Run 2 | 26.21 |
| Deionised Water Run 1 | 34.57 |

As can be seen from the above data and FIG. 17, fluorescence signals generated from the isolation product obtained with the method of the present invention occurred substantially faster than the signal of the negative control. This indicated the successful amplification of isolated RNA.

The above illustrations and accompanying description of the various embodiments of the present invention merely serve to aid in the understanding principle of the said invention. Accordingly, it should not be construed that the present invention described herein is simply limited to the illustrated embodiments of the device and/or apparatus.

What is claimed is:

1. A device for processing a biological and/or chemical sample, the device comprising:
(a) at least one sample processing chamber having an inlet at a first end and a penetrable sealing layer at a second end,
wherein the sealing layer forms at least a part of an inner wall of the sample processing chamber, and
wherein said sealing layer is adapted to seal off the sample processing chamber from the environment until said sealing layer is penetrated to form an outlet; and
(b) an absorption layer, wherein upon penetration of said sealing layer, said absorption layer is in fluid contact with said sealing layer, and is capable of absorbing fluid released from said sample processing chamber, via the outlet.

2. The device according to claim 1, wherein the inlet of the sample processing chamber is sealable.

3. The device according to claims 1 or 2, wherein the first end and the second end of the sample processing chamber are coaxially opposing each other.

4. The device according to any one of claims 1-3, wherein the sealing layer is penetrable by means selected from the group consisting of pressure, mechanical force, moisture, an electrical field, a magnetic field, a change in temperature, a change in pH of the fluid comprised in the sample processing chamber, and any combination thereof.

5. The device according to any one of claims 1-4, wherein the absorption layer is penetrable.

6. The device according to any one of claims 1-5, wherein the absorption layer is contacting the sealing layer.

7. The device according to any one of claims 1-6, comprising a penetrable sealing portion that forms at least a part of a wall of the sample processing chamber, said sealing portion comprising:
(a) said sealing layer penetrable to form an outlet,
wherein said sealing layer has an internal surface and an external surface, wherein said internal surface forms the at least part of an inner wall of the sample processing chamber and the external surface is adapted to seal off the sample processing chamber from the environment until said sealing layer is penetrated to form an outlet; and
(b) said absorption layer, contacting the external surface of said sealing layer, wherein the absorption layer is capable of absorbing fluid released from the chamber, upon penetration of the sealing layer, via the outlet.

8. The device according to any one of claims 1-7, wherein the absorption layer is detachable from the sealing layer.

9. The device of claim 8, wherein the absorption layer is replaceable with at least one other absorption layer.

10. The device of claim 8, wherein the sealing layer is replaceable with at least one other sealing layer.

11. The device according to any one of claims 1-10, wherein the chamber is filled with a medium for carrying out a chemical or biological process.

12. The device according to claim 11, wherein the medium for carrying out a chemical or biological process comprises magnetically attractable particles.

13. The device according to any one of claims 1-12, further comprising a second penetrable sealing layer with the absorption layer sandwiched between said first and second penetrable sealing layers.

14. The device according to any one of claims 1-13, wherein the absorption layer comprises at least two sub-absorption layers of varying absorptivity.

15. The device according to any one of claims 1-14, wherein the absorption layer comprises at least two sub-absorption layers of similar absorptivity.

16. The device according to any one of claims 1-15, wherein the absorption layer is fabricated from a hydrophilic or a hydrophobic material.

17. The device according to claim 16, wherein the hydrophobic material is selected from the group consisting of a cellulose fiber coated with an hydrophobic compound, a perfluoropolyether fiber and a polypropylene fiber.

18. The device according to claim 16, wherein the hydrophilic material is selected from the group consisting of a fluid-absorbing polymer and a cellulose-based material.

19. The device according to claim 18, wherein the cellulose-based material is selected from the group consisting of a wood pulp fiber, a cotton linter, rayon fibers, flax, hemp, jute, ramie and cotton.

20. The device according to claim 18, wherein the fluid-absorbing polymer is selected from the group consisting of foam, a fluid-absorbing polymer powder and a polymer gel.

21. The device according to any one of claims 1-20, wherein the absorption layer has the capacity to absorb fluid of a volume equating to at least the volume of the sample processing chamber.

22. The device according to any one of claims 1-21, wherein the device is adapted to be securable to at least one other device for processing a chemical and/or biological sample, such that the outlet of one device is in fluid communication with another device.

23. The device according to any one of claims 1-22 comprising two, three, four, five or more sample processing chambers.

24. The device according to any one of claims 1-23, further comprising a thermostatic element.

25. The device, or a plurality thereof, according to any one of claims 1-24, wherein the inlet of the sample processing chamber is adapted to receive a probe.

26. The device according to any one of claims 1-25, comprised in a housing.

27. An apparatus for processing a biological and/or chemical sample comprising:
(a) at least one device comprising:
(i) at least one sample processing chamber having an inlet at a first end and a penetrable sealing layer at a second end,
wherein the sealing layer forms at least a part of an inner wall of the sample processing chamber, and
wherein said sealing layer is adapted to seal off the sample processing chamber from the environment until said sealing layer is penetrated to form an outlet; and
(ii) an absorption layer, wherein upon penetration of said sealing layer, said absorption layer is in fluid contact with said sealing layer, and is capable of absorbing fluid released from said sample processing chamber, via the outlet; and
(b) a probe, wherein said probe is adapted to assist in the processing of the sample.

28. The apparatus of claim 27, wherein the assisting in the processing of the sample is selected from the group consisting of selectively attracting target matter from the sample processing chamber, transporting matter into the chamber, removing matter from the sample processing chamber, agitating the sample and regulating the temperature of the sample.

29. The apparatus of claims 27 or 28, wherein the inlet of the sample processing chamber is adapted to receive said probe.

30. The apparatus of any one of claims 27-29, wherein the probe comprises a head that is adapted to selectively attract target matters.

31. The apparatus of any one of claims 27-30, wherein the probe comprises arms, blades, hollow cylindrical structures or other element surrounding the core rod.

32. The apparatus of any one of claims 27-31, wherein the probe is adapted to agitate fluid.

33. The apparatus of claim 31, wherein the arms, blades, hollow cylindrical structures or other elements surrounding the core rod are adapted to protect a part of the probe.

34. The apparatus of claim 31 or 33, wherein the probe is adapted to agitate fluid by means of arms and wherein said arms are adapted to protect the head of the probe.

35. The apparatus of any of claims 27 to 34, wherein the probe has magnetic or electromagnetic properties or electrical charges.

36. The apparatus of any one of claims 27-35, wherein the probe is adapted to introduce the sample into or remove the sample from the sample processing chamber.

37. The apparatus of any of claims 27 to 36, wherein the probe is adapted to create an outlet in the penetrable sealing layer by mechanical means.

38. The apparatus of any one of claims 27-37, wherein the inlet of the sample processing chamber is sealable.

39. The apparatus of any one of claims 27-38, wherein the absorption layer is contacting the sealing layer.

40. The apparatus of any one of claims 27-39, wherein the first end and the second end of the sample processing chamber are opposing each other.

41. The apparatus of any one of claims 27-40, wherein the absorption layer is penetrable.

42. The apparatus of any one of claims 27-41, wherein the absorption layer is detachable from the sealing layer.

43. The apparatus of claim 42, wherein the absorption layer is replaceable with at least one other absorption layer.

44. The apparatus of claim 42, wherein the sealing layer is replaceable with at least one other sealing layer.

45. The apparatus of any one of claims 27-44, wherein said device comprises a penetrable sealing portion that forms at least a part of a wall of said sample processing chamber, said sealing portion comprising:
(a) said sealing layer penetrable to form an outlet,
wherein said sealing layer has an internal surface and an external surface, wherein said internal surface forms the at least part of an inner wall of the sample processing chamber and the external surface is adapted to seal off the sample processing chamber from the environment until said sealing layer is penetrated to form an outlet; and
(b) said absorption layer, contacting the external surface of said sealing layer, wherein the absorption layer is capable of absorbing fluid released from the chamber, upon penetration of the sealing layer, via the outlet.

46. The apparatus of any one of claims 27-45, further comprising an elution chamber.

47. The apparatus of claim 46, wherein the elution chamber is of smaller volume than the sample reaction chamber.

48. The apparatus of claims 46 or 47, wherein the elution chamber is adapted to receive a head of the probe.

49. A method of processing a biological and/or chemical sample comprising:
(a) Providing a device for processing a biological and/or chemical sample comprising:
(i) at least one sample processing chamber having an inlet at a first end and a penetrable sealing layer at a second end,
wherein the sealing layer forms at least a part of an inner wall of the sample processing chamber, and
wherein said sealing layer is adapted to seal off the sample processing chamber from the environment until said sealing layer is penetrated to form an outlet; and
(ii) an absorption layer, wherein upon penetration of said sealing layer, said absorption layer is in fluid contact with said sealing layer, and is capable of absorbing fluid released from said sample processing chamber, via the outlet;
(b) Providing a biological and/or chemical sample;
(c) Placing said sample into the sample processing chamber of the device, thereby forming a reaction mixture; and
(d) Exposing said reaction mixture to a process, thereby forming a product mixture.

50. The method of claim 49, further comprising:
(e) Selectively attracting target matter suspected to be comprised in the product mixture to the surface of a probe; and
(f) Using said target matter, with or without release from the probe, for further use such as for storage, analysis and disposal.

51. A method of processing a biological and/or chemical sample comprising:
(a) Providing an apparatus for processing a biological and/or chemical sample, said apparatus comprising:
(i) at least one device comprising:
at least one sample processing chamber having an inlet at a first end and a penetrable sealing layer at a second end,
wherein the sealing layer forms at least a part of an inner wall of the sample processing chamber, and
wherein said sealing layer is adapted to seal off the sample processing chamber from the environment until said sealing layer is penetrated to form an outlet; and
an absorption layer, wherein upon penetration of said sealing layer, said absorption layer is in fluid contact with said sealing layer, and is capable of absorbing fluid released from said sample processing chamber, via the outlet; and
(ii) a probe adapted to assist in the processing of the sample;
(b) Providing a biological and/or chemical sample;
(c) Placing said sample into the sample processing chamber of said device, thereby forming a reaction mixture;
(d) Exposing said reaction mixture to a process, thereby forming a product mixture;

52. The method of claim 51, wherein the assisting in the processing of the sample is selected from the group consisting of selectively attracting target matter from the sample processing chamber, agitating the sample and regulating the temperature of the sample.

53. The method of claims 51 or 52, further comprising:
(e) Selectively attracting target matter suspected to be comprised in the product mixture to the surface of the probe; and
(f) Using said target matter, with or without release from the probe, for further use such as for storage, detection, analysis and disposal.

54. The method of claims 52 or 53, wherein the selective attraction of target matter is achieved by means selected from the group consisting of electromagnetic fields, electrical charges and physicochemical means.

55. The method of claim 54, wherein the selective attraction of target matter comprises the use of magnetically attractable particles.

56. The method of any one of claims 49 to 55, wherein the target matter or a precursor thereof is suspected or known to be comprised in the biological and/or chemical sample.

57. The method of any one of claims 49 to 56, wherein the sealing layer is penetrated by means selected from the group consisting of pressure, mechanical force, a change in temperature and a change in pH of the fluid comprised in the sample processing chamber, or any combination thereof.

58. The method of claim 57, wherein the mechanical force is exercised by the probe.

59. A method of any one of claims 49 to 58, wherein the sealing layer is penetrated after attracting target matter suspected to be comprised in the product mixture to the surface of the probe.

60. The method of any one of claims 49 to 59, wherein the absorption layer of the device removes the processed starting material remaining in the sample processing chamber.

61. A method of any one of claims 49 to 60, wherein the sample is exposed to a process selected from the group consisting of
(a) a chemical reaction,
(b) a component exchange,
(c) a cell lysis,
(d) an extraction of a molecule from an organism or a part of an organism,
(e) a release of a molecule from an organism, and any combination thereof.

62. The method of claim 61, wherein the chemical reaction is selected from the group consisting of
(a) a protein synthesis,
(b) a nucleic acid synthesis,
(c) a peptide synthesis,
(d) an enzymatic degradation,
(e) an interaction with a binding molecule, and any combination thereof.

63. The method according to any one of claims 49-62, wherein the sample is selected from the group consisting of milk, soil, a blood sample, a serum sample, a plasma sample, a urine sample, a semen sample, a lymphatic fluid sample, a cerebrospinal fluid sample, a milk sample, an amniotic fluid sample, a biopsy sample, a needle aspiration biopsy sample, a cancer sample, a tumour sample, a tissue sample, a cell sample, a cell lysate sample, a virus culture sample, a crude cell lysate sample, a forensic sample, an archaeological sample, an infection sample, a nosocomial infection sample, a production sample, a drug preparation sample, a biological molecule production sample, a protein preparation sample, a lipid preparation sample, a carbohydrate preparation sample, a solution of a nucleotide, a solution of polynucleotide, a solution of a nucleic acid, a solution of a peptide, a solution of a polypeptide, a solution of an amino acid, a solution of a protein, a solution of a synthetic polymer, a solution of a biochemical composition, a solution of an organic chemical composition, a solution of an inorganic chemical composition, a solution of a lipid, a solution of a carbohydrate, a solution of a combinatory chemistry product, a solution of a drug candidate molecule, a solution of a drug molecule, a solution of a drug metabolite, a suspension of a cell, a suspension of a virus, a suspension of a microorganism, and any combination thereof.

64. The method of any one of claims 49-63, wherein the target matter comprises a protein or a nucleic acid comprising an affinity tag.

65. A fluid separation device comprising:
(a) a penetrable sealing layer adapted to form a wall of a sample processing chamber of the device of claim 1; and
(b) an absorption layer, wherein upon penetration of said sealing layer, said absorption layer is in fluid contact with said sealing layer, and is capable of absorbing fluid released from said sample processing chamber.

66. The fluid separation device of claim 65, wherein the absorption layer is fabricated from a hydrophilic or a hydrophobic material.

67. The fluid separation device of claim 66, wherein the hydrophobic material is selected from a group consisting of a cellulose fiber coated with a hydrophobic compound, a perfluoropolyether fiber and a polypropylene fiber.

68. The fluid separation device of claim 66, wherein the hydrophilic material is selected from the group consisting of a fluid-absorbing polymer and a cellulose-based material.

69. The fluid separation device of claim 68, wherein the cellulose-based material is selected from the group consisting of a wood pulp fiber, a cotton linter, rayon fibers, flax, hemp, jute, ramie and cotton.

70. The fluid separation device of claim 68, wherein the fluid-absorbing polymer is selected from the group consisting of foam, a fluid-absorbing polymer powder and a polymer gel.

71. The fluid separation device of any one of claims 65-70, wherein the absorption layer is contacting the sealing layer.

72. The fluid separation device of any one of claims 65-71, adapted to form at least a part of a wall of the sample processing chamber of said device,
said fluid separation device being penetrable, and comprising:
(a) the sealing layer penetrable to form an outlet of said sample processing chamber,
wherein said sealing layer has an internal surface and an external surface, wherein said internal surface forms the at least part of an inner wall of the sample processing chamber and the external surface is adapted to seal off the sample processing chamber from the environment until said sealing layer is penetrated to form an outlet; and
(b) the absorption layer, contacting the external surface of said sealing layer, wherein the absorption layer is capable of absorbing fluid released from the chamber, upon penetration of the sealing layer, via the outlet.
